(19) United States
(12) Patent Application Publication
(10) Pub. No.: US 2013/0197276 A1
(43) Pub. Date: Aug. 1, 2013

(54) ALKOXYLATES OF HYDROGENATED FARNESOLS AND USE THEREOF

(71) Applicants: Wolfgang SPIEGLER, Worms (DE); Kai-Uwe Baldenius, Heidelberg (DE); Michael Triller, Ilvesheim (DE); Juergen Tropsch, Roemerberg (DE)

(72) Inventors: Wolfgang SPIEGLER, Worms (DE); Kai-Uwe Baldenius, Heidelberg (DE); Michael Triller, Ilvesheim (DE); Juergen Tropsch, Roemerberg (DE)

(21) Appl. No.: 13/755,561

(22) Filed: Jan. 31, 2013

Related U.S. Application Data
(60) Provisional application No. 61/592,681, filed on Jan. 31, 2012.

(51) Int. Cl. C07C 43/178; C07C 41/03

(52) U.S. Cl. CPC C07C 43/1785; C07C 41/03
USPC 568/616

(57) ABSTRACT

The present invention relates to new types of farnesol alkoxylates, based on at least partially hydrogenated farnesol; processes for the preparation of these alkoxylates and the use thereof in washing, rinsing, cleaning or finishing compositions, cosmetic compositions, compositions for papermaking, fuel additives and solubilization auxiliaries in aqueous liquid systems.

The present invention relates to new types of farnesol alkoxylates based on at least partially hydrogenated farnesol; processes for the preparation of these alkoxylates and the use thereof in washing, rinsing, cleaning or finishing compositions, cosmetic compositions, compositions for papermaking, fuel additives and solublization auxiliaries in aqueous liquid systems.

### BACKGROUND OF THE INVENTION

The prior art (cf. for example DE 100 64 491, WO 2001/70384, WO 01/70925, WO 01/70661 and WO 02/24624) discloses the preparation and use of fragrance alcohol alkoxylates, such as, for example, farnesol alkoxylates, in washing, rinsing, cleaning and finishing compositions, and also cosmetic preparations and as solubilization auxiliaries. It does not describe the use of at least partially hydrogenated farnesols as starting material for preparing the alkoxylates. The preparation of mixed alkoxylates of short and medium chain length which are composed of blocks of different alkoxylates, where the primary alkoxylate block, i.e. the alkoxylate block bonded to the farnesol, is a propoxylated block, is likewise not described.

WO 2004/006883 discloses skincare compositions based on branched alcohols with a chain length of at least 7 carbon atoms. Alcohols with a chain length of more than 13 carbon atoms are not disclosed. Furthermore, the alcohol derivatives described therein are characterized by a carboxyl-terminated side group that optionally comprises alkoxy groups.

The farnesol alkoxylates known from the prior art still do not completely meet the requirements of practical application in said fields since the triunsaturated side chain is not very chemically stable and can form various degradation products in the presence of atmospheric oxygen. It is therefore an object of the present invention to provide new types of farnesol alkoxylates with an improved profile of properties.

### SUMMARY OF THE INVENTION

The object referred to above was surprisingly achieved by providing farnesol alkoxylates according to the invention of the general formula I in which R is an at least partially hydrogenated farnesyl radical.

### DETAILED DESCRIPTION OF THE INVENTION

### A) Definitions

Unless stated otherwise, the following definitions are to be used within the context of the present invention.

According to the invention, “farnesol” comprises all stereoisomeric forms of this compound. In particular, therefore, the E/E configurations of natural farnesol, but also the E/Z and the Z/E configuration of synthetic farnesols are also comprised. Mixtures of these configurations may also be present. The same also applies to the hydrocarbon radicals derived from farnesol; hereinbelow also referred to as “farnesyl” radicals.

“An at least partially” hydrogenated farnesol or farnesyl radical is understood as meaning compounds or radicals in which one, two or three of the double bonds of the farnesol or of the farnesyl radical have been hydrogenated. Single hydrogenation (hydrogenation of a single double bond) can take place here in any position in the farnesol or farnesyl radical. Double hydrogenations (hydrogenation of two of the three double bonds) can likewise occur in any desired position. The term “at least partially hydrogenated” also comprises mixtures of partially and completely hydrogenated farnesols or partially and completely hydrogenated farnesyl radicals where, in addition, non-hydrogenated farnesol or non-hydrogenated farnesyl radicals may likewise be present.

“Mixtures” of alkoxylates described herein are present when individual components differ with regard to the meaning of the radical R or of the radical (-A-O—)ₓ. Thus, the x values in the mixture can vary, such as e.g. vary by integers from 0 to 100, 1 to 50, or 1 to 20, in particular 2 to 15, 2 to 10 or 2 to 8, such as e.g. by 3, 4, 5, 6, or 7. In particular, mention may be made of “narrow range” alkoxylates with a low degree of distribution. In the mixture, the parameters x, x1 . . . xn can thus on average also assume non-integer values. The same is true for parameter “n” (see e.g. formula (Ia) below).

The stated degrees of alkoxylation (x values) are in particular statistical average values which may be an integer or a fraction for a specific product.

“Low alkylene” stands in particular for C₂₋₁₂-alkylene radicals, i.e. straight-chain or mono- or polybranched hydrocarbon bridging groups having 2 to 12 carbon atoms of the general formula

—CRₐR_{b}—CR_{c}R_{d}—

in which
Rₐ, R_{b}, R_{c} and R_{d} are identical or different and this of the radicals, independently of the others, is H or a C₁ to C₁₀-alkyl radical, which is in particular straight-chain, with the proviso that at least one of the radicals Rₐ, R_{b}, R_{c} and R_{d} is H and the total number of carbon atoms of the low alkylene group does not exceed 12.

In particular, the low alkylene group can be e.g. C₂-C₆-alkylene groups, e.g. selected from —(CH₂)₂—, —CH₂—CH(Met)-, —CH(Met)-CH₂—, —CH(Met)-CH(Met)-, —C(Met)₂-CH₂—, —CH₂—C(Met)₂-, —C(Met)₂-CH(Met)-, —CH(Met)-C(Met)₂-, —CH₂—CH(Et)-, —CH(Et)-CH₂—, —CH(Et)-CH(Et)-, —C(Et)₂-CH₂—, —CH₂—C(Et)₂-, —CH₂—CH(n-Prop)-, —CH(n-Prop)-CH₂—, —CH(n-Prop)-CH(Met)-, —CH₂—CH(n-Bu)-, —CH(n-Bu)-CH₂—, —CH(Met)-CH(Et)-, —CH(Met)-CH(n-Prop)-, —CH(Et)-CH(Met)-, —CH(Met)-CH(Et)-, or is C₂-C₄-alkylene groups, e.g. selected from —(CH₂)₂—, —CH₂—CH(Met)-, —CH(Met)-CH₂—, —CH(Met)-CH(Met)-, —C(Met)₂-CH₂—, —CH₂—C(Met)₂-, —CH₂—CH(Et)-, —CH(Et)-CH₂—, to name but a few nonlimiting examples; where Met is methyl, Et is ethyl, n-Prop is n-propyl and n-Bu is n-butyl.

“Low alkylene oxides” are the corresponding epoxide-bridged compounds of the above “low alkylene” radicals, such as e.g. C₂-C₁₂-, C₂-C₆-, C₂-C₄-, C₃-C₁₀-, or C₃-C₆ alkylene oxide radicals.

“Low alkylene oxide groups” are the radicals (-alkO—) formed therefrom by opening the epoxide bridge.

“Propylene” is in particular groups of the formulae —CH₂—CH(Met)- or —CH(Met)-CH₂—

“Propylene oxide” stands for the corresponding epoxide-bridged compounds of the above “propylene” radicals.

“Propylene oxide groups” are the radicals (—PO—) formed therefrom by opening the epoxide bridge.

“Alkali metal salts” or “Alkali metal hydroxides” comprise in particular salts of litium, sodium and potassium.

“Alkaline earth imetal salts” or “alkaline earth metal hydroxides” comprise in particular salts of magnesium. calcium, strontium and barium.

### B) Specific Embodiments

The present invention relates in particular to the following specific embodiments:
1. Farnesol alkoxylates of the general formula I

R—O-(-A-O—)ₓ—H (I)
in which, simultaneously or independently of one another, in particular simultaneously,
R is an at least partially hydrogenated farnesyl radical,
A is identical or different low alkylene groups, such as e.g. C₂-C₁₂-, C₂-C₆-, or C₂-C₄-alkylene, where the polyoxyalkylene radical (-A-O)ₓ— can consist of one or more different oxyalkylene blocks, and
x is an integer or non-integer from 1 to 100, such as e.g. 2 to 50, 3 to 25, 3 to 15 or 3 to 10; or x is an integer or non-integer of 9 to 100 or 10 to 100 or 11 to 100 or 12 to 100 or 13 to 100, like e.g. 9 to 50, 10 to 25, 12 to 20 or 12 to 18; or mixtures of such farnesol alkoxylates.
In particular, the subject matter is also farnesol alkoxylates of the general formula Ia

R—O-[(-A-O—)ₓ]ₙ—H (Ia)
in which R, A, x are as defined above and n is an integer or non-integer of 2 to 10, like e.g. of 2, 3, 4, 5, 6, 7, 8, 9 or 10; and mixtures of such compounds.
In general, the following apply for formula I and/or formula Ia:
In particular, R can also be completely hydrogenated farnesyl.
In particular, R can also be partially hydrogenated farnesyl.
A can in particular be ethylene.
A can in particular be C₃-alkylene, in particular isopropylene.
A can in particular be C₄-alkylene.
x can be a non-integer or integer, in particular 1, 2, 3, 4, 5, 6, 7, 8, 9, 10, 11, 12, 13, 14, 15 or 16.
n can in a non-integer or integer, particular be 1, 2 or 3.
(-A-O—)ₓ can in particular be an EO block.
(-A-O—)ₓ can in particular be a C₃-alkylene oxide, in particular isopropylene oxide block.
(-A-O—)ₓ can in particular be a C₄-alkylene oxide block.
-[(-A-O—)ₓ]ₙ can in particular be 2 different alkylene oxide blocks.
-[(-A-O—)ₓ]ₙ can in particular be 3 different alkylene oxide blocks.
-[(-A-O—)ₓ]ₙ can in particular comprise one EO block and one C₃-alkylene oxide block, in particular isopropylene oxide block in the order EO-PO or PO-EO.
-[(-A-O—)ₓ]ₙ can in particular comprise one EO block, one C₃-alkylene oxide block or PO block, in particular isopropylene oxide block, and one AlkO block, in the order EO-PO-AlkO or EO-AlkO-PO or PO-EO-AlkO or PO-AlkO-EO or AlkO-EO-PO or AlkO-PO-EO, where, if AlkO is a C₃-alkylene oxide, adjacent blocks comprise different alkylene radicals (in which PO is propylene oxide, in particular isopropylene oxide, EO is ethylene oxide and AlkO is a C₃-C₁₀-alkylene oxide block).
2. Farnesol alkoxylates according to embodiment 1, in which R is a (C₁₅) radical of the formula II

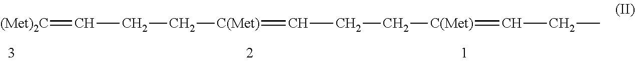

in which
one of the double bonds in positions 1, 2 or 3, or
two of the double bonds in positions 1, 2 or 3, or
all three double bonds in positions 1, 2 and 3,
are hydrogenated; or
in which R is a mixture of such partially or completely hydrogenated radicals, optionally additionally comprising non-hydrogenated farnesyl radicals; or mixtures of such farnesol alkoxylates.
3. Farnesol alkoxylates according to one of the preceding embodiments, in which
   a) the group -(-A-O)ₓ— is a polyoxyalkylene radical,
   in which at the same time or independently of one another, in particular at the same time,
   x is a value from 3 to 50, such as e.g. 3 to 25 or 4 to 10; and
   A is in each case identical low alkylene groups, such as e.g. C₂₋₁₂-, C₂₋₆-, or C₂₋₄-alkylene;
   or
   A has different meanings and is at least two different low alkylene groups, such as e.g. at least two different C₁₋₆-, or C₁₋₄- or C₂₋₆-, or C₂₋₄-alkylene groups which are distributed randomly over the polyoxyalkylene radical;
   -A-O— here is in particular EO, PO or AlkO according to the above definition
   x here is in particular 3 to 25
   x is for example 4, 5, 6, 7, 8, 9 or 10.
   or
   b) the polyoxyalkylene radical -(-A-O)ₓ— is composed of n, such as e.g. 2, 3, 4, 5 or 6, oxyalkylene blocks -(-A₁-O)ₓ₁— to -(-Aₙ-O)ₓₙ—, where x is the sum x1+ . . . +xn and at least two oxyalkylene blocks differ with regard to the meaning of the alkylene radicals (A₁ . . . Aₙ) and/or with regard to the number of their monomer building blocks (x1 . . . xn) and where x1 to xn, independently of one another, are integers from 1 to 50, such as e.g. 2 to 50, 3 to 25, 3 to 15 or 3 to 10;
   n here is in particular 2, 3 or 4.
   x1 to xn, independently of one another, are, for example, 3, 4, 5, 6, 7, 8, 9 or 10. -A-O— here is in particular EO, PO or AlkO according to the above definition,
   or mixtures of such farnesol alkoxylates.
4. Farnesol alkoxylates according to embodiment 3, in which the polyoxyalkylene radical is composed of different oxyalkylene blocks and is selected from the radicals:
   —(PO)ₓ₁-(EO)ₓ₂-H
   —(PO)ₓ₁-(EO)ₓ₂-(AlkO)ₓ₃-H
   in which, simultaneously or independently of one another, in particular simultaneously,
   PO is propylene oxide, EO is ethylene oxide and AlkO is C₃-C₁₀-alkylene oxide,
   x1 is an integer from 1, 2 or 3;
   x2 is an integer from 3 to 50, such as e.g. 3 to 20, 3 to 10 or 4 to 8; and
   x3 is an integer from 1 to 10, such as e.g. 2 to 9, or 5 to 8;
   or mixtures of such farnesol alkoxylates.
5. Farnesol alkoxylates according to embodiment 4, in which, simultaneously or independently of one another, in particular simultaneously,
   x1 is an integer from 1 or 2;
   x2 is an integer from 6 to 9, such as in particular 7 or 8; and
   x3 is an integer from 1 to 5;
   or mixtures of such farnesol alkoxylates.
6. Process for the preparation of a compound of the general formula I

R—O-(-A-O—)ₓ—H (I)
where
a) an alcohol of the general formula III

R—OH (III)
in which R has the meanings given above, is alkoxylated in the presence of an alkoxylation catalyst, in the presence or absence of a solvent and also in the presence of at least one low alkylene oxide or low alkylene oxide mixture, giving a compound of the formula I,
in which the group -(-A-O)ₓ— is a polyoxyalkylene radical, in which, simultaneously or independently of one another, in particular simultaneously,
x is a value from 3 to 50 such as e.g. 3 to 20, 3 to 10 or 4 to 8, and
A is in each case identical low alkylene groups; or
A has different meanings and is at least two different low alkylene groups which are distributed randomly over the polyoxyalkylene radical;
or
b) an alcohol of the general formula III

R—OH (III)
in which R has the meanings given above, is alkoxylated in the presence of an alkoxylation catalyst, in the presence or absence of a solvent and in the presence of a first low alkylene oxide, and the alkoxylation is repeated (n−1) times changing the low alkylene oxide, where n is an integer (such as e.g. from 1 to 10), giving a compound of the formula I in which the polyoxyalkylene radical -(-A-O)ₓ— is composed of n oxyalkylene blocks -(-A₁-O)ₓ₁— . . . -(-Aₙ-O)ₓₙ—,
where x is the sum x1+ . . . +xn and at least two oxyalkylene blocks differ with regard to the meaning of the alkylene radicals (A₁ . . . Aₙ) and/or with regard to the number of their monomer building blocks (x1 . . . xn) and where x1 to xn, independently of one another, are integers from 1 to 50, such as e.g. 3 to 20, 3 to 10 or 4 to 8.
7. Process according to embodiment 6 b), where
   i) the alcohol of the above formula III is propoxylated in the presence of an alkali metal or alkaline earth metal hydroxide, such as NaOH or KOH, or of a multimetal cyanide catalyst and in the presence or absence of a solvent and in the presence of propylene oxide to give a propoxide of the formula IV

R—O—(PO)ₓ₁—H (IV)
and then
ii) the propoxylate formed in this way is further alkoxylated by adding one of the following blocks
-(EO)ₓ₂- or
-(EO)ₓ₂-(AlkO)ₓ₃-;
where x1 to x3, independently of one another, are integers from 1 to 50, such as e.g. 3 to 20, 3 to 10 or 3 to 8.
Stage i) and ii) can be carried out here with or preferably without catalyst change.
8. Process according to embodiment 7, in which the multimetal cyanide catalyst is selected from catalysts which have at least one dimetal cyanide compound of the general formula (D)

M²ₐ[M¹(CN)ᵣXₜ]_{b}
in which
M¹ is a metal ion from the group consisting of Zn(II), Fe(II), Co(III), Ni(II), Mn(II), Co(II), Sn(II), Pb(II), Fe(III), Mo(IV), Mo(VI), Al(III), V(IV), V(V), Sr(II), W(IV), W(VI), Cu(II) and Cr(III),
M² is a metal ion from the group consisting of Sr(I), Mg(II), Zn(II), Fe(II), Fe(III), Co(III), Cr(III), Mn(II), Mn(III), Ir(III), Rh(III), Ru(II), V(IV), V(V), Co(II), Cr(II), Ti(IV),
X is a group different from cyanide which forms a coordinative bond to M¹, selected from the group consisting of carbonyl, cyanate, isocyanate, nitrite, thiocyanate and nitrosyl,
a, b, r, t are integers which are selected such that the electroneutrality condition is satisfied.
9. Process according to embodiment 8, where M¹ is Zn(II) and M² is selected from Co(III) and Fe(III).
10. Use of at least one compound according to one of embodiments 1 to 5 in washing, rinsing, cleaning or finishing compositions; in cosmetic compositions; in fuels or fuel additive mixtures in agrochemical compositions; or in compositions for papermaking.
11. Composition selected from washing, rinsing, cleaning or finishing compositions; cosmetic compositions; compositions for papermaking; agrochemical compositions or fuels or fuel additive mixtures, comprising at least one compound according to one of the embodiments 1 to 5.
12. Method, selected from washing, rinsing, cleaning or finishing methods; methods for papermaking; or methods for producing agrochemical compositions; fuels or fuel additive mixtures, where at least one compound according to one of embodiments 1 to 5 is used in this method.
13. Use of a compound according to one of embodiments 1 to 5 as solubilizer in aqueous systems/liquids, such as in particular in emulsion polymerization.
14. Another aspect of the invention refers to a reaction product as obtained by a process of one of the embodiments 6 to 9 and its use as defined above.

### C) Preparation of the Compounds

The farnesol alkoxylates according to the invention of the formula (I) can be prepared in a manner known per se and using customary equipment.

For example, a suitable preparation process starts from the reaction of an at least partially hydrogenated farnesol in a reactor, which is reacted here under suitable conditions with at least one alkylene oxide, i.e. with a single alkylene oxide, a mixture of alkylene oxides or stepwise with identical or different alkylene oxides, until the desired chain length and/or alkylene oxide block sequence is achieved.

Before carrying out the reaction, it may be expedient to dewater the reaction mixture by applying a subatmospheric pressure and optionally applying elevated temperature, such as, for example, a temperature of from 80 to 150° C., e.g. 0.1 to 2 h or 0.5 to 1 h. Furthermore, it may be expedient to render the reaction mixture inert by flushing it with nitrogen.

The reaction usually takes place in the presence of a suitable alkoxylation catalyst, such as, e.g. an alkali metal hydroxide, or a double metal cyanide catalyst suspended in a suitable inert organic solvent, such as e.g. tridecanol N, at a temperature in the range from room temperature to about 200° C., such as, for example, 100 to 180° C. or 120 to 160° C. The reaction can take place here at atmospheric pressure or a superatmospheric pressure of up to 100 bar, such as e.g. at a pressure in the range from 1 to 50 or 1.5 to 20 or 2 to 10 bar.

For example, the molar ratio of at least partially hydrogenated farnesol to alkylene oxide can be in the range from 1:3 to 1:100, such as e.g. 1:4 to 1:20 or 1:5 to 1:10.

The reaction time of the actual alkoxylation can then be in the range from one minute to 20 h, such as e.g. 10 min to 5 h. Following completion of the reaction and neutralization, for example by adding acetic acid, the product can be removed from the reactor.

### D) Uses

The farnesyl alkoxylates according to the invention are used in various “compositions” or “preparations”. Here, these terms are to be understood in the widest sense and comprise preparations in solid form (particles, powders etc.), semisolid form (pastes etc.), liquid form (solutions, emulsions, suspensions, gels, etc.) and gas-like form (aerosols etc.) which, with regard to an advantageous effect of the farnesyl alkoxylates, usually comprise further components which are customary for the particular intended use.

### D1) Washing, Rinsing, Cleaning and Finishing Compositions

The aforementioned farnesyl alkoxylates according to the invention can be added according to the invention to washing, rinsing, cleaning and finishing compositions.

These usually comprise one or more of the farnesyl alkoxylates according to the invention in an amount of from 0.001 to 10% by weight, such as e.g. in an amount of from 0.01 to 5% by weight, or in an amount of from 0.02 to 3% by weight or 0.05 to 2% by weight, based on the total weight of the washing, rinsing, cleaning or finishing composition.

Additionally, further ingredients may be present which further improve the use properties and/or esthetic properties of the washing, rinsing, cleaning and finishing compositions.

For example, one or more substances selected from enzymes, surfactants and also the group of builders, complexing agents, bleaches, bleach activators, electrolytes, nonaqueous solvents, pH extenders, fragrances, perfume carriers, fluorescent agents, dyes, hydrotopes, foam inhibitors, silicone oils, antiredeposition agents, dispersants, optical brighteners, graying inhibitors, shrink preventers, crease protection agents, color transfer inhibitors, antimicrobial active ingredients, antioxidants, corrosion inhibitors, antistats, ironing aids, phobicization and impregnation agents, swelling and nonslip agents and also UV absorbers may be present as further ingredients.

Complexing agents are e.g. methylglycinediacetic acid (MGDA), ethylendiaminetriacetic acid (EDTA), nitrilotriacetic acid (NTA), diethylenetriamine-pentaacetic acid (DTPA), tartaric acid, citric acid, glucuronic acid and glycolic acid, and also the salts of these compounds.

The enzymes are preferably selected from hydrolases, such as proteases, esterases, glucosidases, lipases, amylases, cellulases, mannanases, pectate lyases, other glcosylhydrolases and mixtures of the aforementioned enzymes. All of these hydrolyases contribute during washing to the removal of stains such as protein-, fat- or starch-containing stains and graying. Cellulases and other glycosyihydrolases can, moreover, contribute to retaining the color and increasing the softness of the textile by removing pilling and microfibrils. Oxireductases can also be used for bleaching and/or for preventing color transfer. Enzymatic active ingredients obtained from bacteria strains or fungi such as Bacillus subtilis, Bacillus licheniformis, Streptomyceus griseus and Humicola insolens are of particularly good suitability.

Suitable hydrolases are e.g. α-glucosidases (EC number 3.2.1.20), β-glucosidases (Ovozyme; EC number 3.2.1.21), amylases (Purastar, Termamyl, Stainzyme, Duramyl), mannanases (Purabrite, Mannastar, Mannaway) and cellulases (Carezyme, Celluzyme, Endolase, Puradax). Suitable amylases include in particular α-amylases (EC number 3.2.1.1), isoamylases and pullulanases. Furthermore, mention is to be made of glycosidases (EC number 3.2.1.15), such as pectinases and pectate lyases (EC number 4.2.2.2). The cellulases used are preferably cellobiohydrolases, endoglucanases and 3-glucosidases, which are also called cellobiases, or mixtures of these. Since different cellulosase types differ in their CMCase and avicelase activities, the desired activities can be established by targeted mixtures of the cellulases

Suitable lipases are esterases, such as lipex and lipolase. Examples of lipolytically active enzymes are also the known cutinases.

Peroxidases or oxidases have also proven to be suitable in some cases.

For example, an enzyme mixture may also be present. Preference is given, for example, to enzyme mixtures which comprise the following enzymes or consist of them:
protease and amylase,
protease and lipase (or lipolytically active enzymes),
protease and cellulase,
amylase, cellulase and lipase (or lipolytically active enzymes),
protease, amylase and lipase (or lipolytically active enzymes)
protease, lipase (or lipolytically active enzymes) and cellulase.

Preferred proteases in the aforementioned mixtures are proteases of the subtilisin type (Savinase, etc.; EC number 3.4.21.62).

The enzymes can be adsorbed to carrier substances in order to protect them against premature decomposition. The fraction of enzymes is preferably 0.1 to 5% by weight, particularly preferably 0.15 to 2.5% by weight, in particular 0.2 to 2% by weight, based on the total weight of the detergent or cleaner composition.

Surfactants which can be used are anionic, nonionic, cationic and/or amphoteric surfactants, as well as mixtures e.g. of anionic and nonionic surfactants. The total surfactant content in the composition can be 5 to 60% by weight, such as 15 to 40% by weight, based on the total weight of the composition.

The nonionic surfactants used are e.g. alkoxylated, advantageously ethoxylated, in particular primary alcohols having preferably 8 to 18 carbon atoms and on average 1 to 20, such as 1 to 12, mol of ethylene oxide (EO) per mole of alcohol, in which the alcohol radical can be linear or preferably methyl-branched in the 2 position or can comprise linear and methyl-branched radicals in a mixture, as are usually present in oxo alcohol radicals. In particular, however, alcohol ethoxylates with linear radicals from alcohols of native origin having 12 to 18 carbon atoms, for example from coconut alcohol, palm alcohol, tallow fatty alcohol or oleyl alcohol, and on average 2 to 8 EO per mole of alcohol are preferred. Preferred ethoxylated alcohols include, for example, C₁₂-C₁₄-alcohols with 3 EO, 4 EO or 7 EO, C₉-C₁₁-alcohols with 7 EO, C₁₃-C₁₅-alcohols with 3 EO, 5 EO, 7 EO or 8 EO, C₁₂-C₁₈-alcohols with 3 EO, 5 EO or 7 EO and mixtures of these, such as mixtures of C₁₂-C₁₄-alcohol with 3 EO and C₁₂-C₁₈-alcohol with 7 EO. Furthermore, mention may be made of the corresponding alkoxylates of Guerbet alcohols, such as in particular 2-propylheptanol. The stated degrees of ethoxylation are statistical average values which may be an integer or a fraction or a specific product. Alcohol ethoxylates which have a narrowed homolog distribution (narrow range ethoxylates, NRE) are also suitable. In addition to these nonionic surfactants, it is also possible to use fatty alcohols with more than 12 EO. Examples thereof are tallow fatty alcohol with 14 EO, 25 EO or 30 EO. Nonionic surfactants which comprise EO and PO groups together in the molecule can also be used. In this connection, it is possible to use block copolymers with EO-PO block units or PO-EO block units, but also EO-PO-EO copolymers or PO-EO-PO copolymers. It is of course also possible to use mixed alkoxylated nonionic surfactants in which EO and PO units are not blockwise, but in random distribution. Such products are obtainable through the simultaneous action of ethylene oxide and propylene oxide on fatty alcohols.

Moreover, further nonionic surfactants which can be used are also alkyl glycosides of the general formula (1)

R¹O(G)ₓ (1)

in which R¹ is a primary straight-chain or methyl-branched, in particular 2-methyl-branched aliphatic radical having 8 to 22, preferably 10 to 18, carbon atoms, and G is a glycoside unit having 5 or 6 carbon atoms, preferably glucose. The degree of oligomerization x, which indicates the distribution of monoglycosides and oligoglycosides, is any desired number between 1 and 10; preferably, x is 1.2 to 1.4.

A further class of preferably used nonionic surfactants which are used either as the sole nonionic surfactant or in combination with other nonionic surfactants are alkoxylated, preferably ethoxylated or ethoxylated and propoxylated fatty acid alkyl esters, preferably having 1 to 4 carbon atoms in the alkyl chain, in particular fatty acid methyl esters, as are described, for example, in the Japanese patent application JP 58/217598 or which are preferably prepared by the method described in the international patent application WO-A-90/13533.

Nonionic surfactants of the amine oxide type, for example N-cocosalkyl-N,N-dimethylamine oxide and N-tallowalkyl-N,N-dihydroxyethylamine oxide, and of the fatty acid alkanolamides may also be suitable. The amount of these nonionic surfactants is preferably not more than that of the ethoxylated fatty alcohols, in particular not more than half thereof.

Further suitable surfactants are polyhydroxy fatty acid amides of the formula (2),

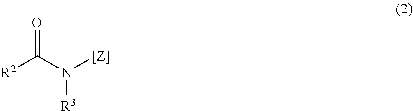

in which R²C(═O) is an aliphatic acyl radical having 6 to 22 carbon atoms, R³ is hydrogen, an alkyl or hydroxyalkyl radical having 1 to 4 carbon atoms and [Z] is a linear or branched polyhydroxyalkyl radical having 3 to 10 carbon atoms and 3 to 10 hydroxyl groups. The polyhydroxyl fatty acid amides are known substances which can usually be obtained by reductive amination of a reducing sugar with ammonia, an alkylamine or an alkanolamine and subsequent acylation with a fatty acid, a fatty acid alkyl ester or a fatty acid chloride.

The group of polyhydroxy fatty acid amides also includes compounds of the formula (3)

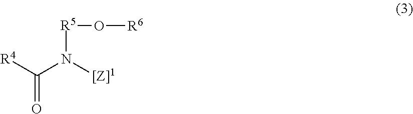

in which R⁴ is a linear or branched alkyl or alkenyl radical having 7 to 12 carbon atoms,

R⁵ is a linear, branched or cyclic alkylene radical having 2 to 8 carbon atoms or an arylene radical having 6 to 8 carbon atoms and R⁶ is a linear, branched or cyclic alkyl radical or an aryl radical or an oxyalkyl radical having 1 to 8 carbon atoms, where C₁-C₄-alkyl or phenyl radicals are preferred, and [Z]¹ is a linear polyhydroxyalkyl radical whose alkyl chain is substituted with at least two hydroxyl groups, or alkoxylated, preferably ethoxylated or propoxylated derivatives of this radical. [Z]¹ is preferably obtained by reductive amination of a sugar, for example glucose, fructose, maltose, lactose, galactose, mannose or xylose. The N-alkoxy- or N-aryloxy-substituted compounds can then be converted to the desired polyhydroxy fatty acid amides for example in accordance with WO-A-95/07331 by reaction with fatty acid methyl esters in the presence of an alkoxide as catalyst.

The content of nonionic surfactants in the liquid detergents or cleaners is preferably 0 to 30% by weight, preferably 0 to 20% by weight and in particular 2 to 15% by weight, in each case based on the total weight of the detergent or cleaner composition.

The anionic surfactants used are, for example, those of the sulfonate and sulfate types. Suitable surfactants of the sulfonate type are preferably C₉-C₁₃-alkylbenzenesulfonates, olefinsulfonates, i.e. mixtures of alkene- and hydroxyalkanesulfonates, and also disulfonates, as are obtained, for example, from C₁₂-C₁₈-monoolefins with terminal or internal double bond by sulfonation with gaseous sulfur trioxide and subsequent alkaline or acidic hydrolysis of the sulfonation products. Also suitable are alkanesulfonates which are obtained from C₁₂-C₁₈-alkanes, for example by sulfochlorination or sulfoxidation with subsequent hydrolysis or neutralization. Likewise, the esters of α-sulfo fatty acids (estersulfonates), for example the α-sulfonated methyl esters of the hydrogenated coconut, palm kernel or tallow fatty acids, are also suitable.

Further suitable anionic surfactants are sulfated fatty acid glycerol esters. Fatty acid glycerol esters are to be understood as meaning the mono-, di- and triesters, and mixtures thereof, as are obtained in the preparation by esterification of a monoglycerol with 1 to 3 mol of fatty acid or during the transesterification of triglycerides with 0.3 to 2 mol of glycerol. Preferred sulfated fatty acid glycerol esters here are the sulfonation products of saturated fatty acids having 6 to 22 carbon atoms, for example of caproic acid, caprylic acid, capric acid, myristic acid, lauric acid, palmitic acid, stearic acid or behenic acid.

Preferred alk(en)yl sulfates are the alkali metal and in particular the sodium salts of the sulfuric acid half-esters of C₁₂-C₁₈-fatty alcohols, for example of coconut fatty alcohol, tallow fatty alcohol, lauryl alcohol, myristyl alcohol, cetyl alcohol or stearyl alcohol or of the C₁₀-C₂₀-oxo alcohols and those half-esters of secondary alcohols of these chain lengths. Furthermore, preference is given to alk(en)yl sulfates of the specified chain length which comprise a synthetic, petrochemical-based straight-chain alkyl radical which have an analogous degradation behavior to the equivalent compounds based on fatty chemical raw materials. From a washing point of view, the C₁₂-C₁₆-alkyl sulfates and C₁₂-C₁₅-alkyl sulfates and also C₁₄-C₁₅-alkyl sulfates are preferred. 2,3-alkyl sulfates, which are prepared, for example, in accordance with the U.S. Pat. Nos. 3,234,258 or 5,075,041 and can be obtained as commercial products from the Shell Oil Company under the name DAN®, are also suitable anionic surfactants.

The sulfuric acid monoesters of the straight-chain or branched C₇-C₂₁-alcohols ethoxylated with 1 to 6 mol of ethylene oxide, such as 2-methyl-branched C₉-C₁₁-alcohols having on average 3.5 mol of ethylene oxide (EO) or C₁₂-C₁₈-fatty alcohols with 1 to 4 EO, are also suitable. On account of their high foaming behavior, they are used in cleaners only in relatively small amounts, for example in amounts from 1 to 5% by weight.

Further suitable anionic surfactants are also the salts of alkylsulfosuccinic acid, which are also referred to as sulfosuccinates or as sulfosuccinic acid esters and which constitute monoesters and/or diesters of sulfosuccinic acid with alcohols, preferably fatty alcohols and in particular ethoxylated fatty alcohols. Preferred sulfosuccinates comprise C₈-C₁₈-fatty alcohol radicals or mixtures of these. Particularly preferred sulfosuccinates comprise a fatty alcohol radical which is derived from ethoxylated fatty alcohols. In this connection, particular preference is given in turn to sulfosuccinates whose fatty alcohol radicals are derived from ethoxylated fatty alcohols with a narrow homolog distribution. It is likewise also possible to use alk(en)ylsuccinic acid having preferably 8 to 18 carbon atoms in the alk(en)yl chain or salts thereof.

Particularly preferred anionic surfactants are soaps. Saturated and unsaturated fatty acid soaps, such as the salts of lauric acid, myristic acid, palmitic acid, stearic acid, (hydrogenated) erucic acid and behenic acid, and also in particular soap mixtures derived from natural fatty acids, for example coconut, palm kernel, olive oil or tallow fatty acids, are suitable.

The anionic surfactants including the soaps can be present in the form of their sodium, potassium or ammonium salts, and also as soluble salts of organic bases, such as mono-, di- or triethanolamine. The anionic surfactants are preferably present in the form of their sodium or potassium salts, in particular in the form of the sodium salts.

The content of anionic surfactants (including the soaps) is e.g. 2 to 50% by weight, such as about 3 to 40% by weight, in each case based on the total weight of the composition.

The viscosity of liquid compositions can be measured using customary standard methods (for example Brookfield viscosimeter LVT-II at 20 rpm and 20° C., spindle 3) and is preferably in the range from 100 to 5000 mPas. Preferred compositions have viscosities of from 300 to 4000 mPas, with values between 1000 and 3000 mPas being particularly preferred.

Builders which may be present in the compositions are, in particular, silicates, aluminum silicates (in particular zeolites) and carbonates, and also mixtures of these substances. The use of such builders, however, is not preferred.

A broad number of highly diverse salts can be used as electrolytes from the group of inorganic salts. Preferred cations are the alkali metal and alkaline earth metals, preferred anions are the halides and sulfates. From the point of view of production, the use of NaCl or MgCl₂ in the compositions is preferred. The fraction of electrolytes is usually 0.5 to 5% by weight, based on the total weight of the composition.

The composition according to the invention or the composition used for the use according to the invention can comprise at least one solvent. Suitable solvents are selected from water, nonaqueous solvents and mixtures thereof. The nonaqueous solvents used are preferably nonaqueous organic solvents. Preferred nonaqueous organic solvents are those which are completely miscible with water under standard conditions (20° C., 1013 mbar).

The solvent content of the detergent or cleaner composition is preferably 5 to 95% by weight, particularly preferably 10 to 80% by weight, based on the total weight of the composition.

Nonaqueous solvents which can be used originate, for example, from the group of mono- or polyhydric alcohols, alkanolamines or glycol ethers, provided they are miscible with water in the stated concentration range. Preferably, the solvents are selected from ethanol, n- or isopropanol, butanols, glycol, propane- or butanediol, glycerol, diglycol, propyl or butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl, ethyl or propyl ether, dipropylene glycol monomethyl or ethyl ether, diisopropylene glycol monomethyl or ethyl ether, methoxy-, ethoxy- or butoxytriglycol, isobutoxyethoxy-2-propanol, 3-methyl-3-methoxybutanol, propylene glycol t-butyl ether, and mixtures of these solvents. Nonaqueous solvents can be used in amounts between 0.5 and 15% by weight, but preferably below 12% by weight and in particular below 9% by weight.

In order to bring the pH of the liquid composition into the desired range, the use of pH extenders may be appropriate. All known acids or alkalis can be used here provided their use is not precluded for applications-related or ecological reasons or for reasons of consumer protection. Usually, the amount of these extenders does not exceed 7% by weight, based on the total weight of the detergent or cleaner composition.

In order to improve the esthetic impression of the compositions, they can be colored with suitable dyes. Preferred dyes, the selection of which presents no difficulty at all to the person skilled in the art, have a high storage stability and insensitivity toward the other ingredients of the compositions and to light, and also no marked substantivity toward textile fibers, in order not to stain these.

Suitable foam inhibitors which can be used are, for example, soaps, paraffins or silicon oils, which can optionally be applied to carrier materials.

Suitable antiredeposition agents, which are also referred to as “soil repellents”, are, for example, nonionic cellulose ethers, such as methylcellulose and methylhydroxypropylcellulose with a fraction of methoxy groups of 15 to 30% by weight and of hydroxypropyl groups of 1 to 15% by weight, in each case based on the nonionic cellulose ethers. Suitable soil release polymers are, for example, polyesters of polyethylene oxides with ethylene glycol and/or propylene glycol and aromatic dicarboxylic acids or aromatic and aliphatic dicarboxylic acids; polyesters of polyethylene oxides that are terminally capped at one end with di- and/or polyhydric alcohols and dicarboxylic acid, in particular polymers of ethylene terephthalates and/or polyethylene glycol terephthalates or anionically and/or nonionically modified derivatives of these. Of these, particular preference is given to the sulfonated derivatives of the phthalic acid and terephthalic acid polymers. Polyesters of this type are known, for example, from U.S. Pat. No. 3,557,039, GB-A 11 54 730, EP-A 0 185 427, EP-A 0 241 984, EP-A 0 241 985, EP-A 0 272 033 and U.S. Pat. No. 5,142,020. Further suitable soil release polymers are amphiphilic graft polymers or copolymers of vinyl esters and/or acrylic esters on polyalkylene oxides (cf. U.S. Pat. No. 4,746,456, U.S. Pat. No. 4,846,995, DE-A 37 11 299, U.S. Pat. No. 4,904,408, U.S. Pat. No. 4,846,994 and U.S. Pat. No. 4,849,126) or modified celluloses such as e.g. methylcellulose, hydroxypropylcellulose or carboxymethylcellulose.

Optical brighteners (so-called “whiteners”) can be added to the compositions in order to eliminate graying and yellowing of the treated textile sheet materials. These substances attach to the fibers and bring about a brightening and quasi bleaching effect by converting invisible ultraviolet radiation into visible longer-wave light, where the ultraviolet light absorbed from the sunlight is emitted as pale bluish fluorescence and produces pure white with the yellow shade of grayed and/or yellowed laundry. Suitable compounds originate for example from the substance classes of the 4,4′-diamino-2,2′-stilbenedisulfonic acids (flavonic acids), 4,4′-distyrylbiphenylene, methylumbelliferones, coumarins, dihydroquinolinones, 1,3-diarylpyrazolines, naphthalimides, benzoxazole, benzisoxazole and benzimidazole systems, and the pyrene derivatives substituted by heterocycles. The optical brighteners are usually used in amounts between 0.03 and 0.3% by weight, based on the finished composition.

Dispersants, such as in particular polycarboxylates (e.g. SOKALANE from BASF SE) are to be mentioned as further suitable additives.

Graying inhibitors have the task of keeping the dirt detached from the fibers suspended in the liquor and thus preventing reattachment of the dirt. Of suitability for this purpose are water-soluble colloids mostly of an organic nature, for example size, gelatins, salts of ethersulfonic acids of starch or of cellulose or salts of acidic sulfuric acid esters of cellulose or of starch. Water-soluble polyamides comprising acidic groups are also suitable for this purpose. Furthermore, soluble starch preparations and starch products other than those mentioned above can be used, for example degraded starch, aldehyde starches etc. Polyvinylpyrrolidone can also be used. However, preference is given to using cellulose ethers, such as carboxymethylcellulose (Na salt), methylcellulose, hydroxyalkylcellulose and mixed ethers such as methylhydroxyethylcellulose, methylhydroxypropylcellulose, methylcarboxymethyl-cellulose and mixtures thereof in amounts of 0.1 to 5% by weight, based on the composition.

Since textile sheet materials, in particular made of rayon, viscose rayon, cotton and mixtures thereof have a tendency to crease because the individual fibers are sensitive to bending, folding, pressing and squeezing at right angles to the fiber direction, the compositions can comprise synthetic crease protection agents. These include, for example, synthetic products based on fatty acids, fatty acid esters, fatty acid amides, fatty alkylol esters, fatty alkylol amides or fatty alcohols, which are mostly reacted with ethylene oxide, or products based on lecithin or modified phosphoric acid esters.

To control microorganisms, the compositions can comprise antimicrobial active ingredients. A distinction is made here, depending on the antimicrobial spectrum and action mechanism, between bacteriostats and bactericides, fungistats and fungicides, germicides etc. Important substances from these groups are, for example, benzalkonium chlorides, alkylarylsulfonates, halophenols and phenol mercuriacetate.

In order to prevent undesired changes in the compositions and/or the treated textile sheet materials caused by the effect of oxygen and other oxidative processes, the compositions can comprise antioxidants. This class of compound includes, for example, substituted phenols, hydroquinones, pyrocatechins and aromatic amines, and also organic sulfides, polysulfides, dithiocarbamates, phosphites and phosphonates.

Increased wear comfort can result from the additional use of antistats which are additionally added to the compositions. Antistats increase the surface conductivity and thus permit an improved discharging of charges formed. External antistats are generally substances with at least one hydrophilic molecule ligand and produce a more or less hygroscopic film on the surfaces. These mostly interface-active antistats can be divided into nitrogen-containing antistats (amines, amides, quaternary ammonium compounds), phosphorus-containing antistats (phosphoric acid esters) and sulfur-containing antistats (alkylsulfonates, alkyl sulfates). External antistats are described, for example, in the patent applications FR 1,156,513, GB 873 214 and GB 839 407. The lauryl (or stearyl) dimethylbenzylammonium chlorides disclosed here are suitable as antistats for textile sheet materials and as additive to detergents, in which case a hand-modifying effect is additionally achieved.

To improve the water absorption capacity, the rewettability of the treated textile sheet materials and to facilitate ironing of the treated textile sheet materials, silicone derivatives, for example, can be used in the compositions. These additionally improve the rinse-out behavior of the compositions by virtue of their foam-inhibiting properties. Preferred silicone derivatives are, for example, polydialkyl- or alkylarylsiloxanes in which the alkyl groups have one to five carbon atoms and are partially or completely fluorinated. Preferred silicones are polydimethylsiloxanes which can, optionally, be derivatized and then are amino functional or quaternized or have Si—OH, Si—H and/or Si—Cl bonds. The viscosities of the preferred silicones at 25° C. are in the range between 100 and 100 000 mPas, it being possible to use the silicones in amounts between 0.2 and 5% by weight, based on the total composition.

Finally, the compositions can also comprise UV absorbers, which attach to the treated textile sheet materials and improve the photostability of the fibers. Compounds which have these desired properties are, for example, the compounds and derivatives of benzophenone with substituents in the 2 and/or 4 position that are effective as a result of nonradiative deactivation. Furthermore, substituted benzotriazoles, acrylates phenyl-substituted in the 3 position (cinnamic acid derivatives), optionally with cyano groups in the 2 position, salicylates, organic Ni complexes, and also natural substances such as umbelliferon and the endogenous urocanic acid are also suitable.

Liquid compositions have no sediment and can be e.g. transparent or at least translucent. For example, the aqueous liquid compositions have a visible light transmission of at least 30%, preferably 50%, particularly preferably 75%, most preferably 90%. Alternatively, the thickneners according to the invention can be incorporated into opaque detergents or cleaners.

Besides these constituents, an aqueous composition can comprise dispersed particles, the diameter of which along their largest spatial expansion is 0.01 to 10 000μm.

Particles may be microcapsules as well as granules, compounds and scented beads, with microcapsules being preferred.

The term “microcapsules” is understood as meaning aggregates which comprise at least one solid or liquid core which is surrounded by at least one continuous shell, in particular a shell made of polymer(s). Usually, these are finely dispersed liquid or solid phases surrounded by film-forming polymers, during the production of which the polymers, following emulsification and coacervation or interfacial polymerization, precipitate onto the material to be enveloped. The microscopically small capsules can be dried like powders. Besides single-core microcapsules, multicore aggregate, also called microspheres, are also known and comprise two or more cores distributed in the continuous shell material. Single-core or multicore microcapsules can moreover be surrounded by an additional second, third etc. shell. Preference is given to single-core microcapsules with a continuous shell. The shell can consist of natural, semisynthetic or synthetic materials. Natural shell materials are, for example, gum Arabic, agar agar, agarose, maltodextrins, alginic acid or salts thereof, e.g. sodium alginate or calcium alginate, fats and fatty acids, cetyl alcohol, collagen, chitosan, lecithins, gelatin, albumin, schellac, polysaccharides, such as starch or dextran, sucrose and waxes. Semisynthetic shell materials are, inter alia, chemically modified celluloses, in particular cellulose esters and ethers, e.g. cellulose acetate, ethylcellulose, hydroxpropylcellulose, hydropropylmethylcellulose and carboxymethylcellulose, and also starch derivatives, in particular starch ethers and esters. Synthetic coating materials are, for example, polymers such as polyacrylates, polyamides, polyvinyl alcohol or polyvinylpyrrolidone. In the interior of the microcapsules, sensitive, chemically or physically incompatible and also volatile components (=active ingredients) of the aqueous liquid detergent or cleaner can be enclosed in a storage-stable and transport-stable manner. For example, optical brighteners, surfactants, complexing agents, bleaches, bleach activators, dyes and fragrances, antioxidants, builders, enzymes, enzyme stabilizers, antimicrobial active ingredients, graying inhibitors, antiredeposition agents, pH extenders, electrolytes, foam inhibitors and UV absorbers may be present in the microcapsules.

The microcapsules can also comprise catonic surfactants, vitamins, proteins, preservatives, detergency boosters or pearlizing agents. The fillings of the microcapsules can be solids or liquids in the form of solutions or emulsions or suspensions.

The microcapsules can have any desired form within the scope of manufacture, but are preferably approximately spherical. Their diameter along their largest spatial expansion can be between 0.01 μm (not visually recognizable as capsules) and 10 000 μm, depending on the components present in their interior and the application. Preference is given to visible microcapsules with a diameter in the range from 100 μm to 7000 μm, in particular from 400 μm to 5000 μm. The microcapsules are accessible by known methods, with coacervation and interfacial polymerization being attributed the greatest importance. Microcapsules which can be used are all of the surfactant-stable microcapsules supplied on the market, for example the commercial products (the coating material is given in each case in brackets) Hallcrest Microcapsules (gelatin, gum arabic), Coletica Thalaspheres (maritimes collagen), Lipotec Millicapseln (alginic acid, agar agar), Induchem Unispheres (lactose, microcrystalline cellulose, hydroxypropylmethylcellulose); Unicerin C30 (lactose, microcrystalline cellulose, hydroxypropylmethylcellulose), Kobo Glycospheres (modified starch, fatty acid esters, phospholipids), Softspheres (modified agar agar) and Kuhs Probiol Nanospheres (phospholipids).

Alternatively, it is also possible to use particles which do not have a core-shell structure but in which the active ingredient is distributed in a matrix made of a matrix-forming material. Such particles are also referred to as “speckles”.

A preferred matrix-forming material is alginate. To produce alginate-based speckles, an aqueous alginate solution, which also comprises the active ingredient to be enclosed or the active ingredients to be enclosed, is dripped and then hardened in a precipitation bath comprising Ca²⁺ ions or Al³⁺ ions.

Alternatively, instead of alginate, other matrix-forming materials can be used. Examples of matrix-forming materials comprise polyethylene glycol, polyvinylppyrrolidone, polymethacrylate, polylysine, poloxamer, polyvinyl alcohol, polyacrylic acid, polyethylene oxide, polyethoxyoxazoline, albumin, gelatin, acacia, chitosan, cellulose, dextran, Ficoll®, starch, hydroxyethylcellulose, hydroxypropylcellulose, hydroxypropylmethylcellulose, hyaluronic acid, carboxymethylcellulose, carboxymethylcellulose, deacetylated chitosan, dextran sulfate and derivates of these materials. The matrix formation takes place for these materials for example via gelling, polyanion-polycation interactions or polyelectrolyte-metal ion interactions. The preparation of particles with these matrix-forming materials is known per se.

The particles can be stably dispersed in the aqueous liquid detergents or cleaners. Stable means that the compositions are stable at room temperature and at 40° C. over a period of at least 4 weeks and preferably of at least 6 weeks without the compositions creaming up or sedimenting. The thickeners according to the invention bring about, through the increase in viscosity, a kinetic slowing of the sedimentation of the particles and thus their stabilization in the suspended state.

The release of the active ingredients from the microcapsules or speckles usually takes place during the application of the compositions comprising them as a result of destruction of the shell or of the matrix as a result of mechanical, thermal, chemical or enzymatic action.

The compositions according to the invention can be used for the cleaning of textile sheet materials and/or hard surfaces. Cleaners according to the invention can be present in the form of a hand or machine dishwashing detergent, all-purpose cleaner for non-textile surfaces, e.g. made of metal, painted wood or plastic, or cleaner for ceramic articles, such as porcelain, tiles. Preferably, the detergents or cleaners according to the invention are present in the form of a liquid textile detergent. These may be formulated in solid, liquid or pasty form.

### D2) Cosmetic Compositions

Preparations according to the invention can also be cosmetic preparations which differ in their composition from washing, rinsing, cleaning and finishing compositions according to the preceding description. This applies in particular to non-surfactant-containing cosmetic preparations. Also comprised according to the invention are, however, surfactant-containing cosmetic preparations. Preferably, these cosmetic compositions comprise farnesyl alkoxylates in an amount of 0.001 to 10% by weight, preferably in an amount of 0.01 to 5% by weight, such as in an amount of 0.02 to 3 or in an amount of 0.05 to 2% by weight, in each case based on the cosmetic composition.

The cosmetic compositions are for example aqueous preparations which optionally comprise surfactants or surface-active ingredients and which are suitable in particular for treating keratin fibers, in particular human hair, or for treating the human skin.

For the most important ingredient group, the surface-active (surfactant) active ingredients or washing active ingredients, predominantly fatty alcohol polyglycolether sulfates (ether sulfates, alkyl ether sulfates) are used, sometimes in combination with other, mostly anionic surfactants. Apart from a good cleaning power and insensitivity toward water hardness, shampoo surfactants should have skin and mucosa compatibility. A good biodegradability should be present corresponding to the legal provisions. As well as the alkyl ether sulfates, the compositions can additionally comprise further surfactants, such as alkyl sulfates, alkyl ether carboxylates, preferably with degrees of ethoxylation of 4 to 10, and also surface-active protein-fatty acid condensates. Here, the protein-abietic acid condensate in particular is to be mentioned. Sulfosuccinic acid esters, amidopropylbetaines, amphoacetates and amphodiacetates and also alkyl polyglycosides are also surfactants used preferably in hair shampoos.

A further group of ingredients is summarized under the term auxiliaries and is very diverse: for example, additives of nonionic surfactants such as ethoxylated sorbitan esters or of protein hydrolysates increase the compatibility and/or have an irritation-reducing effect, e.g. in baby shampoos; refatting agents for preventing excessive degreasing during hair washing are e.g. natural oils or synthetic fatty acid esters; humectants are glycerol, sorbitol, propylene glycol (see propanediols), polyethylene glycols and other polyols. To improve the wet combability and reduce electrostatic charging of the hair after drying, cationic surfactants such as e.g. quaternary ammonium compounds can be added to the shampoos. For a colored, brilliant appearance, dyes and/or pearlescent pigments are added. To establish the desired viscosity, thickeners of different substance classes can be used, a pH stability is achieved by buffers, e.g. based on citrate, lactate or phosphate. In order to ensure adequate shelf life and storability, preservatives, such as e.g. 4-hydroxybenzoic acid esters, are added; oxidation-sensitive ingredients can be protected by adding antioxidants such as ascorbic acid, butylmethoxyphenol or tocopherol.

A third group of ingredients is formed by specific active ingredients for special shampoos, e.g. oils, herb extracts, proteins, vitamins and lecithins in shampoos for hair which becomes greasy quickly, for especially dry hair and for stressed or damaged hair. Active ingredients in shampoos for controlling dandruff in most cases have a broad growth-inhibiting effect against fungi and bacteria. In particular, the fungistat properties e.g. of pyrithion salts have been demonstrated as being the reason for a good antidandruff effect. To produce a pleasant scent note, the hair shampoos comprise perfume oils and/or fragrances. In this connection, all customary fragrances permitted in hair shampoos can be used.

Hair care compositions have the aim of retaining the natural state of freshly regrown hair for as long a period as possible and, in the event of damage, of restoring it. Features which characterize this natural state are silky shine, low porosity, vigorous and at the same time soft fullness and pleasant smooth feel. An important prerequisite for this is a clean, flake-free and not excessively greasy scalp. Haircare compositions nowadays include a large number of different products, the most important representatives of which are referred to as pretreatment compositions, hair tonics, styling aids, hair rinses and treatment packs, and the composition of which is divided, as in the case of the hair washing compositions, roughly into basic materials, auxiliaries and specific active ingredients.

The basic materials are fatty alcohols, primarily cetyl alcohol (1-hexadecanol) and stearyl alcohol (1-octadecanol), waxes such as bees wax, wool wax (lanolin), spermaceti and synthetic waxes, paraffins, Vaseline, paraffin oil and also, as solvents, primarily ethanol, 2-propanol and water. Auxiliaries are emulsifiers, thickeners, preservatives, antioxidants, dyes and perfume oils. The most important group of specific active ingredients in the hair care compositions nowadays is the quaternary ammonium compounds. A distinction is made between monomeric quaternary ammonium compounds [e.g. alkyltrimethylammonium halide with primarily the lauryl, cetyl or stearyl group as alkyl radical) and polymeric quaternary ammonium compounds (e.g.: quaternary cellulose ether derivatives or poly(N,N-dimethyl-3,4-methylenepyrrolidinium chloride)]. Their effect in hair care compositions is based on the fact that the positive charge of the nitrogen atoms in this compound can position itself on the negative charges of the hair keratin; on account of its higher cysteic acid content, damaged hair comprises more negatively charged acid groups and can therefore accommodate more quaternary ammonium compounds. These, also referred to on account of their cation-active character as “cation-active care substances” have a smoothing effect on the hair, improve the combability, reduce the electrostatic charging, improve feel and shine. The polymeric quaternary ammonium compounds adhere so well to the hair that their effect can still be detected after several washes. Organic acids such as citric acid, tartaric acid or lactic acid are often used for establishing an acidic medium. On account of their close chemical similarity, the water-soluble protein hydrolysates attach readily to the hair keratin. The largest group of specifically active ingredients in hair care compositions is formed by various plant extracts and plant oils, which in most cases have already been used for a long time without their effectiveness on the attributed effect having been scientifically demonstrated beyond doubt in all cases. In precisely the same way, the effectiveness of vitamins used in hair care compositions has only been demonstrated in isolated cases. To avoid rapid regreasing, some hair tonics comprise substances such as certain tar ingredients, cysteic acid derivatives or glycyrrhizin; the envisaged reduction in sebaceous gland production has likewise still not been definitively proven. By contrast, the effectiveness of antidandruff active ingredients has been demonstrated beyond doubt. They are therefore used in corresponding hair tonics and other care compositions.

The aqueous preparations for treating skin are in particular preparations for caring for human skin. This care starts with cleaning, for which soaps are primarily used. A distinction is made here between solid, mostly bar-shaped, and liquid soap. Accordingly, the cosmetic preparations in a preferred embodiment are in the form of shaped bodies which comprise surface-active (surfactant) ingredients.

In a preferred embodiment, the most important ingredients of such shaped bodies are the alkali metal salts of the fatty acids of natural oils and fats, preferably with chains from 12 to 18 carbon atoms. Since lauric acid soaps foam particularly well, the lauric acid-rich coconut and palm kernel oils are preferred raw materials for the manufacture of fine soap. The Na salts of the fatty acid mixtures are solid, the K salts are soft-pasty. For the saponification, the diluted sodium hydroxide solution or potassium hydroxide solution is added to the fatty raw materials in the stoichiometric ratio such that an alkali excess of max. 0.05% is present in the finished soap. Often, the soaps are nowadays no longer manufactured directly from the fats, but from the fatty acids obtained by fat cleavage. Customary soap additives are fatty acids, fatty alcohols, lanolin, lecithin, vegetable oils, partial glycerides and other fat-like substances for refatting the cleansed skin, antioxidants such as ascorbyl palmitate or tocopherol for preventing the autoxidation of the soap (rancidity), complexing agents such as nitrilotriacetate for binding traces of heavy metal, which could catalyze the autoxidative deterioration, perfume oils for achieving the desired scent notes, dyes for coloring the soap bars and optionally special additives.

The most important types of fine soaps are: toilet soaps with 20 to 50% coconut oil in the fatty mixture, up to 5% refatting fraction and 0.5 to 2% perfume oil, they form the largest proportion of the fine soaps; luxury soaps with up to 5% of sometimes particularly expensive perfume oils; deodorant soaps with additions of deodorizing active ingredients, such as e.g. 3,4,4′-trichlorocarbanilide (triclocarban); cream soaps with particularly high proportions of refatting and skin-creaming substances; baby soaps with good refatting and additionally care fractions such as e.g. chamomile extracts, at best very weakly perfumed; skin protection soaps with high fractions of refatting substances and also further care and protecting additives, such as e.g. proteins; transparent soaps with additions of glycerin, sugar etc., which prevent the crystallization of the fatty acid salts in the solidified soap melt and thus bring about a transparent appearance; floating soaps with a density <1, created by air bubbles incorporated in a controlled manner during manufacture.

Soaps can also be provided with abrasive additives for cleaning heavily soiled hands. Upon washing with soap, a pH of 8 to 10 is established in the wash liquor. This alkalinity neutralizes the natural acid mantel of the skin (pH value 5 to 6). Although this is reformed relatively quickly in the case of normal skin, it can lead to irritations in the case of sensitive or predamaged skin. A further disadvantage of soaps is the formation of insoluble lime soaps in hard water. These disadvantages are not present in the case of syndet soaps. Their basis is synthetic anionic surfactants which can be processed with builder substances, refatting agents and further additives to give soap-like bars. Their pH is variable within wide limits and is in most cases adjusted to be neutral at pH 7 or adapted to the acid mantel of the skin at pH 5.5. They have exceptional cleaning power, lather at every water hardness, even in seawater, the proportion of refatting additives must be significantly higher than in the case of normal soaps on account of their intensive cleaning and degreasing effect. Their disadvantage is the relatively high price.

Liquid soaps are based either on K salts of natural fatty acids or on synthetic anionic surfactants. They comprise fewer washing-active substances than solid soaps in aqueous solution, have the customary additives, optionally with viscosity-regulating constituents and also pearlescent additives.

On account of their convenient and hygienic application from dispensers, they are preferentially used in public washrooms and the like.

Washing lotions for particularly sensitive skin are based on mild-acting synthetic surfactants with additives of skincare substances, adjusted to be pH neutral or weakly acidic (pH 5.5).

For cleansing primarily the facial skin there is a series of further preparations, such as face tonics, cleansing lotions, milk, creams, pastes; face packs serve sometimes for cleansing, but predominantly for refreshing and care of the facial skin. Facial tonics are mostly aqueous-alcoholic solutions with low surfactant fractions and also further skincare substances. Cleansing lotions, milk, creams and pastes are based mostly on O/W emulsions with relatively small contents of fatty components with cleaning and care additives. So-called scruffing and peeling preparations comprise mildly keratolytic substances for removing the uppermost dead horny layers of the skin, sometimes with additives of abrasive powders. Almond bran, used for a long time as a mild skin cleanser, is still nowadays often a constituent of such preparations. In compositions for the cleaning treatment of blemished skin, moreover, antibacterial and anti-inflammatory substances are present since the sebum accumulations in comedones (blackheads) are breeding grounds for bacterial infections and tend toward inflammations. The broad pallet of different skin cleansing products on offer varies in composition and content of diverse active ingredients, matched to the various skin types and to specific treatment aims.

The bathing additives supplied for skin cleansing in the bath or shower have found broad application. Bathing salt and bathing tablets are intended to soften, color and perfume the bathing water and generally comprise no washing-active substances. By softening the bathing water, they promote the cleaning power of soaps, but are primarily intended to have a refreshing effect and enhance the bathing experience. The foam baths are of greater importance. In the case of a higher content of refatting and skincare substances, the term “cream baths” is also used.

The skincare which follows cleansing has two essential aims: firstly, it is intended to return to the skin the ingredients such as horny cells, skin fat lipids, acid formers and water, removed uncontrollably during washing into the natural equilibrium state, and secondly it is intended to counteract the natural aging process of the skin and the possible damage caused by weathering and environmental influences as far as possible. Preparations for skincare and for skin protection are supplied in large numbers and in many preparation forms. The most important are skin creams, lotions, oils and skin gels. The basis of the creams and lotions is emulsions in O/W (oil in water) or W/O (water in oil) form. The main constituents of the oil- or fat- or lipid-phase are fatty alcohols, fatty acids, fatty acid esters, waxes, Vaseline, paraffins and also further fat and oil components primarily of natural origin. In the aqueous phase, as well as water, primarily moisture-regulating and moisture-retaining substances are present as essential skincare active ingredients, and also consistency- or viscosity-regulating agents. Further additives such as preservatives, antioxidants, complexing agents, perfume oils, colorants and also special active ingredients are added to one of the two aforementioned phases depending on their solubility and their stability properties. The selection of the emulsifier system is essential for this type of emulsion and its properties. It can be selected in accordance with the HLB system.

According to their area of application, the creams and lotions can be divided into day creams and night creams. Day creams are mostly formulated as O/W emulsions, they absorb rapidly into the skin without leaving behind a greasy shine; they are therefore also sometimes referred to as dry creams, matte creams or vanishing creams. Night creams are mostly W/O emulsions, they are absorbed more slowly by the skin and often comprise special active ingredients which are intended to bring about skin regeneration during the night rest. Some of these preparations are also referred to as nutrient creams although nourishment of the cell metabolism in the skin can only take place via the blood circulation; the term nutrient cream is therefore disputed. So-called cold creams are mixed emulsions of the O/W and W/O type, with the oil phase being predominant in terms of amount. In the classic cold cream, upon application, the water, which was in part only emulsified in an unstable way, became free and produced a cooling effect as a result of evaporation, which gave this preparation form its name.

It is not possible to discuss here each of the multitude of special active ingredients used in the skincare compositions and the effects attributed to them. Mention may be made of milk protein products, egg yolk, lecithins, lipods, phosphatides, cereal germ oils, vitamins—in particular vitamin F and biotin, referred to previously as skin vitamin (vitamin H), and also hormone-free placenta extracts. Hormones sometimes used previously are no longer used since they are classified as drug active ingredients and must not be used in cosmetic compositions.

Skin oils belong to the oldest product forms of skincare and are still used nowadays. Their basis is non-drying plant oils such as almond oil or olive oil, with additions of natural vitamin oils such as wheatgerm oil or avocado oil, and also oily plant extracts from e.g. St John's wort, chamomile and the like.

The addition of antioxidants to combat randicity is imperative, desired scent notes are achieved by means of perfume oils, an addition of paraffin oil or liquid fatty acid esters serves to optimize the application properties.

Skin gels are semisolid transparent products which are stabilized by corresponding gel formers. A distinction is made between oleogel (anhydrous), hydrogels (oil-free) and oil/water gels. The type selection is determined by the desired application purpose. The oil/water gels comprise high emulsifier fractions and have certain advantages over emulsions, both from esthetic and application perspectives.

Foot baths are intended to have a cleansing, refreshing, circulation-promoting and invigorating and deodorizing and horny-skin-softening effect. Foot bath additives are available as bath salt and foam baths. They consist e.g. of basic mixtures of sodium carbonate, sodium hydrogencarbonate and sodium perborate or sodium hexametaphosphate, sodium sulfate, sodium perborate and 1% sodium lauryl sulfate as foam component with antihydrotic, deodorizing, optionally bactericidal and/or fungicidal additives and also fragrances and dyes. Foot powders, applied after washing the feet and/or sprinkled into stockings and shoes, are intended to have a skin-smoothing, cooling, moisture-absorbing, perspiration-inhibiting, antiseptic, deodorizing and optionally horny-skin-softening effect. They generally consist of up to 85% of talc with additives of silica powder, aluminum hydroxychloride, salicylic acid and also optionally bactericides, fungicides, deodorants and fragrances. Foot cream and foot balsams are used for skincare and also for massaging the foot and lower leg muscles. Foot creams are generally O/W emulsions of e.g. 30% isopropyl myristate, 10% polysorbate, 4.2% aluminum metahydroxide and 55.8% water as the basic formulation; foot balsams are mostly anhydrous and comprise e.g. 85% Vaseline, 5% paraffin, 3% lanolin, 3% methyl salicylate, 2% camphor, 1% menthol and 1% eucalyptus oil. Hard skin removers such as e.g. “rubbing creams” are rubbed on the skin until the horny skin is removed in the form of small bits. A guide formulation consists of 25% paraffin, 2% stearic acid, 2% beeswax, 2%, spermaceti, 2% glycerol monostearate, 0.5% 2,2′,2″-nitrilotriethanol, 1% perfume oil, 0.2% 4-hydroxybenzoic acid and 65.3% water. Nail fold tinctures are used to soften cornification in the nail folds and to keep the edges of in growing toenails soft, primarily on the big toes.

A guide formulation is made up of 10%, 2,2′,2″-nitrilotriethanol, 15% urea, 0.5% fatty alcohol polyglycol ether and 74.5% water. Further cosmetic compositions preferred according to the invention are compositions for influencing body odor. In particular, deodorizing compositions are intended here. Deodorants of this type can conceal, remove or destroy odors. Unpleasant body odors are formed during the bacterial decomposition of perspiration, in particular in the damp warm armpits where microorganisms find good living conditions. Accordingly, the most important ingredients of deodorants are germ-inhibiting substances. Particular preference is given to those germ-inhibiting substances which have largely selective effectiveness against the bacteria responsible for body odor. However, preferred active ingredients only have a bacteriostatic effect and by no means kill off all of the bacterial flora. Germ-inhibiting agents are generally all suitable preservatives with a specific action against Gram-positive bacteria. These are, for example, Irgasan DP 300 (trichlosan, 2,4,4′-trichloro-2′-hydroxydiphenyl ether), chlorhexidin (1,1′-hexamethylenebis(5-(4′-chlorophenyl)biguanide) and also 3,4,4′-trichlorocarbanilide. Quaternary ammonium compounds are also likewise suitable in principle. On account of their high antimicrobial effectiveness, all of these substances are preferably used only in low concentrations of about 0.1 to 0.3% by weight. Furthermore, numerous fragrances also have antimicrobial properties. Accordingly, fragrances of this type having antimicrobial properties are preferably used in deodorants. In particular, farnesol and phenoxyethanol are to be mentioned here. It is therefore particularly preferred if the deodorants according to the invention comprise such per se bacteriostatically effective fragrances. The fragrances can preferably be present in the form of fragrance alcohol alkoxylates. However, it is also possible that antibacterially effective fragrances are used together with fragrance alcohol alkoxylates and are thus present in mixtures with other fragrances. A further group of essential ingredients of deodorants are enzyme inhibitors which inhibit the decomposition of the perspiration by enzymes, such as, for example, triethyl citrate or zinc glycinate. Furthermore, essential ingredients of deodorants are also antioxidants, which are intended to prevent an oxidation of the perspiration constituents.

In a further likewise preferred embodiment of the invention, the cosmetic composition is a hair-arranging composition which comprises polymers for the setting. It is particularly preferred here if at least one polyurethane is present among the polymers.

In this connection, the compositions according to the invention can, in a preferred embodiment, comprise water-soluble polymers from the group of nonionic, anionic, amphoteric and zwitterionic polymers.

Water-soluble polymers here are to be understood as meaning those polymers which are soluble at room temperature in water in more than 2.5% by weight.

Water-soluble polymers preferred according to the invention are nonionic. Suitable nonioniogenic polymers are, for example: polyvinylpyrrolidones, as are sold, for example, under the name Luviskol® (BASF). Polyvinylpyrrolidones are preferred nonionic polymers for the purposes of the invention.
Vinylpyrrolidone/vinylester copolymers, as are sold, for example, under the trade name Luviskol® (BASF). Luviskol® VA 64 and Luviskol® VA 73, both vinylpyrrolidone/vinyl acetate copolymers, are particularly preferred nonionic polymers.
Cellulose ethers, such as hydroxypropylcellulose, hydroxyethylcellulose and methylhydroxypropylcellulose, as are sold, for example, under the trade names Culminal® and Benecel® (AQUALON).

Suitable amphoteric polymers are, for example, the octylacrylamide/methyl methacrylate/tert-butylaminoethyl methacrylate/2-hydroxypropyl methacrylate copolymers available under the names Amphomer® and Amphomer® LV-71 (DELFT NATIONAL).

Suitable zwitterionic polymers are, for example, the polymers disclosed in the German patent applications DE 39 29 973, DE 21 50 557, DE 28 17 369 and DE 37 08 451. Acrylamidopropyltrimethylammonium chloride/acrylic acid or methacrylic acid copolymers and the alkali metal and ammonium salts thereof are preferred zwitterionic polymers. Further suitable zwitterionic polymers are methacroylethylbetaine/methacrylate copolymers, which are commercially available under the name Amersette® (AMERCHOL).

Anionic polymers suitable according to the invention are inter alia:
Vinyl acetate/crotonic acid copolymers, as are commercially available, for example, under the names Resyn® (NATIONAL STARCH), Luviset® (BASF) and Gafset® (GAF).
Vinylpyrrolidone/vinyl acrylate copolymers, obtainable for example under the trade name Luviflex® (BASF). A preferred polymer is the vinylpyrrolidone/acrylate terpolymer available under the name Luviflex® VBM-35 (BASF).
Acrylic acid/ethyl acrylate/N-tert-butylacrylamide terpolymers as are sold, for example, under the name Ultrahold® strong (BASF).

In cases where the polyurethane comprises ionic groups, it has proven expedient if further water-soluble polymers are nonionogenic or of identical ionogenicity.

Hair-treatment compositions according to the invention comprise water-soluble polymers preferably in amounts of 0.01 to 20% by weight, in particular 0.1 to 10% by weight, based on the total composition, depending on the type of hair-treatment composition, which is not subject to any limitations.

The polyurethanes and the water-soluble polymers are preferably present in the compositions according to the invention in a quantitative ratio of 1:10 to 10:1. A quantitative ratio of 2:1 to 1:1 has proven to be particularly suitable in many cases.

The hair-arranging compositions according to the invention are in particular hair-setting compositions, hair sprays and blow-waving compositions. Hair sprays are a particularly preferred embodiment of the hair-arranging compositions according to the invention.

Furthermore, in a likewise preferred embodiment, the compositions according to the invention can also be formulated as a foam aerosol using a propellant.

The preparations according to the invention which comprise one or more alkoxylated fragrance alcohols exhibit an optimized scent impression on account of the content of the alkoxylated fragrance alcohols. Thus, in preparations for which the alkoxylated fragrance alcohols produced in the way described in this application have been distilled prior to being added to the preparation and have thus been freed from the fragrance alcohol bringing about the scent (the alkoxylated fragrance alcohol was virtually odorless prior to the addition), the alkoxylated fragrance alcohols bring about a scent that already characterizes the preparation which lasts for a prolonged period and thus also survives a relatively long storage time or transportation time of the (optionally surfactant-containing) preparation. Moreover, the scent is readily transferred to the object to be treated, where it adheres reliably; it is released over a prolonged period and thus leads in a reliable manner to the desired scent note. This can be referred to as surprising both for detergents and cleaners and also for cosmetic preparations.

Fragrances are added in particular to surfactant-containing preparations in order to improve the esthetic overall impression of the products and to provide the consumer with a sensory typical and unmistakeable product as well as the technical effect (washing, rinsing, cleaning result). In a preferred embodiment of the present invention, one or more further components that leave behind a scent impression can be added to the surfactant-containing preparations alongside the alkoxylated fragrance alcohols. Perfume oils or fragrances which can be used are individual fragrance compounds, for example the synthetic products of the ester, ether, aldehyde, ketone, alcohol and hydrocarbon types.

Fragrance compounds of the ester type are, for example, benzyl acetate, phenoxyethyl isobutyrate, p-t-butylcyclohexyl acetate, linalyl acetate, dimethylbenzylcarbinyl acetate, phenylethyl acetate, linalyl benzoate, benzyl formate, ethyl methyl phenyl glycinate, allyl cyclohexylpropionate, styrallyl propionate, cyclohexyl salicylate, benzyl salicylate, floramat, melusate and jasmacyclate. Also to be mentioned here are the esters of fragrance alcohols with inorganic acids or organic acids, as are disclosed in the prior art specified at the start.

The ethers include, for example, benzyl ethyl ether and ambroxan. The aldehydes include e.g. linear alkanals having 8 to 18 carbon atoms, citral, citronellal, citronellyloxyacetaldehyde, cyclamenaldehyde, hydroxycitronellal, lilial and bourgeonal.

The ketones include the ionones, α-isomethylionone, and methyl cedryl ketone.

The alcohols include anethole, citronellol, eugenol, geraniol, linalool, phenylethyl alcohol and terpineol. The hydrocarbons include primarily terpenes such as limonene and pinene.

Preference is given to using mixtures of different fragrances which are matched to one another such that they together produce a pleasant scent note.

Such perfume oils can also comprise natural fragrance mixtures, as are accessible from plant sources. Natural fragrances which may be mentioned are: extracts from flowers (lily, lavender, linden blossom, orange blossom, chamomile, rose, jasmine, neroli, ylang ylang), stems and leaves (geranium, patchouli, petitgrain, sage, Melissa, mint, cinnamon leaves), fruits (anise, coriander, cumin, mace, cloves, juniper), fruit peels (bergamot, lemon, oranges), roots (mace, angelica, celery, cardamom, costus, iris, calmus, vetiver), woods (pine wood, sandalwood, guaiac wood, cedar wood, rose wood), herbs and grasses (tarragon, lemongrass, sage, thyme), needles and branches (spruce, fir, pine, dwarf-pine), resins and balsams (galbanum, elemi, benzoin, myrrh, olibanum, opoponax, labdanum). Animal raw materials are also suitable, such as, for example, civet and castoreum.

Usually, the content of fragrances is in the range of 2% by weight of the total preparation.

These can be incorporated directly into the washing-active preparation; however, it may also be advantageous to apply the fragrances to carriers which increase the adhesion of the perfume to the laundry and, by virtue of a slower release of scent, ensure a long-lasting scent on the textiles. Cyclodextrins, for example, have proven to be useful as such carrier materials. In this connection, the cyclodextrin-perfume complexes can additionally also be coated with further auxiliaries.

### D3) Compositions for Papermaking

A further area of application of farnesyl alkoxylates according to the invention is papermaking. In the pulp-containing mixtures used for this purpose (as a rule aqueous suspensions of pulps, which can sometimes also originate from the processing of waste paper), usually one or more of the farnesyl alkoxylates according to the invention are present in an amount of from 0.001 to 5% by weight, such as e.g. in an amount of 0.01 to 4% by weight, or in an amount of 0.02 to 3% by weight or 0.05 to 2% by weight, based on the total weight of the mass, and/or are added to these from the papermaking.

An essential constituent of such pulp compositions is usually a chemical pulp which has been obtained by chemical destructurization of a lignocellulose material such as wood. Examples of chemical pulps include sulfate pulp, sulfite pulp and/or soda pulps, where the pulp may be bleached or unbleached. Among the bleached chemical pulps, chlorine-bleached or in particular low-chlorine or chlorine-free pulps, such as ECF pulp and TCF pulp, are used. Preference is given to an unbleached chemical pulp. Also suitable is chemical pulp from annual plants, such as, for example, chemical pulp based on rice, wheat, sugarcane (bagasse), bamboo or kenaf. Typically, the fraction of chemical pulp in the pulp composition is in the range from 10 to 80% by weight, often in the range from 20 to 70% by weight, based on the total pulp mass in the pulp composition. Sometimes, this can also be replaced by other pulps, such as e.g. waste paper pulp. Further fiber constituents comprise wood pulp, such as, for example, groundwood (=mechanical pulp), e.g. white or brown mechanical pulp, thermomechanical pulp (TMP), chemothermalmechanical pulp (CTMP), semichemical pulp, high yield chemical pulp and refiner mechanical pulp (RMP).

Additionally, further customary additives can be used. Examples of customary additives are the additives customary in paper production for improving and/or modifying the paper properties, such as fillers, sizing agents, wet and dry strength enhancers, antiblocking agents, flame retardants, antistats, hydrophobicizers, dyes and optical brighteners and also process chemicals, such as retention, flocculation and drainage aids, fixatives, mucilage control agents, wetters, defoamers, biocides and the like.

Examples of customary wet strength agents are the polyamides, epichlorohydrin resins, melamine-formaldehyde resins and cationic glyoxylated polyacrylamides usually used for this purpose.

Examples of customary dry strength agents are: native starches, starch derivatives, dextrans, cationized starch, cationically glyoxylated polyacrylamides, polyvinylamines, cationic, anionic or amphoteric polyacrylamides and also mixtures thereof with inorganic dry strengthening agents.

Examples of sizing agents (internal and surface sizing agents) are rosin sizes, casein and comparable proteins, starch, polymer dispersions, reactive sizes, such as, in particular, alkyldiketenes and alkylsuccinic anhydrides.

In addition, the aqueous pulp suspension may also have added to it customary fillers insofar as these have not already been introduced via the waste paper materials. Examples of suitable fillers are, in particular, calcium carbonate such as chalk, kaolin, titanium dioxide, gypsum, precipitated calcium carbonate, talc, silicates.

Examples of typical retention aids are aluminum sulfate and polyaluminum chlorites. Retention aids which can be used are also microparticle systems of high molecular weight polyacrylamides and bentonite or colloidal silica. Retention aids which can be used are also combinations of microparticle systems of high molecular weight polyacrylamides and bentonite or colloidal silica with an anionic organic polymer, in particular anionic, optionally crosslinked polyacrylamides. Retention aids based on microparticle systems of this type are known, for example, from EP 462365, WO 02/33171, WO 01/34908 or WO 01/34910. Retention aids which can be used are also partially hydrolyzed homopolymers of N-vinylformamide and also partially hydrolyzed copolymers of N-vinylformamide with diallyldimethylammonium chloride, N,N-dimethylaminoethylacrylamide, N,N-dimethylaminopropylacrylamide. The retention aids are also microparticle systems of high molecular weight polyvinylamines and anionic, cationic or amphoteric, crosslinked polyacrylamides, as are known, for example, from US 2003/0192664 A1.

Examples of customary flocculation and dewatering aids are polyethyleneimines, polyamines with molar masses of more than 50 000, polyamidoamines which are optionally crosslinked by grafting with ethyleneimine and subsequent crosslinking with e.g. polyethylene glycol dichlorohydrin ethers or with epichlorohydrin, polyetheramines, polyvinylimidazoles, polyvinylimidazolines, polyvinyltetrahydropyridines, polydialkylaminoalkyl vinyl ethers, polydialkylaminoalkyl(meth)acrylates in protonated or quaternized form, polydiallyldialkylammonium halides, such as, in particular, polydillyldimethylammonium chloride.

Examples of customary fixatives are: aluminum sulfate, polyaluminum chlorites, and also cationic polymers customary for this purpose, e.g. cationic polyacrylamides, polyethyleneimines, polyvinylamines, polyimidazolines, polyimidazoles, polyamines, dicyandiamide resins, poly-DADMAC, Mannich products and Hofmann products.

The type and amount of process chemicals and fillers is governed in a manner known per se by the requirements of the paper machine and of the desired type of paper.

The fiber suspension is dewatered in a paper machine to form paper and/or card. Optionally, the fiber suspension can be diluted with water before being introduced (so-called thin pulp). The addition of the process chemicals can take place both prior to and after dilution.

The fiber mass is then dewatered in the usual manner to form a sheet. The dewatering is typically carried out in a paper machine in which the customary steps of paper formation are carried out, i.e. sheet formation in the wire section, compaction and/or compression to remove the majority of the water in the press section, drying in the drying section, glazing by calendering and optionally supercalendering. Optionally, the drying section can also comprise a size press in which the paper is treated with a thin-liquid size liquor for surface consolidation. Optionally, the paper machine can also comprise a coater in which the paper is coated with a coating slip. An overview of customary processes for paper production can be found in Roempp, Lexikon Chemie, 10^{th} edition, Thieme Verlag Stuttgart, 1998, p. 3110 to 3115, and also in Ulmann's Encyclopedia of Industrial Chemistry, 5th edition on CD-ROM (R-PAT, Paper and Pulp, Wiley-VCH 1997).

Typical paper types comprise:

Writing papers, i.e. filler-containing and fully sized papers with glazed surfaces which typically have a basis weight in the range from 30 to 80 g/m² and a filler content in the range from 5 to 30% by weight and the surfaces of which are generally coated;
printing papers, i.e. papers which are uncoated or coated and are suitable for printing, which typically have a basis weight in the range from 40 to 150 g/m² and a filler content of up to 20% by weight;
news (print) papers which typically have a basis weight in the range from 38 to 50 g/m² and can have a filler content in the region of up to 18% by weight;
wrapping papers which typically have a basis weight in the range from 70 to 250 g/m² and have a filler content of up to 15% by weight;
card or solid board which typically have a basis weight in the range from 250 to 1000 g/m² and can have a filler content of up to 15% by weight.

### D4) Fuel Additives

According to the invention, the aforementioned farnesyl alkoxylates according to the invention can also be used as fuel additives individually or in a mixture with further fuel additives (in the form of a so-called additive packet).

The fuel additized with the farnesyl alkoxylate according to the invention is a gasoline fuel or a middle distillate fuel, in particular a diesel fuel. In this connection, the compounds according to the invention are usually used in amounts of 0.00001 to 10% by weight or 0.0001 to 5% by weight or 0.001 to 2% by weight or 0.01 to 1% by weight, based on the total weight of the additivized fuel.

The fuel can comprise further customary additives.

In the case of diesel fuels, these are primarily customary detergent additives, carrier oils, cold flow improvers, lubricity improvers, corrosion inhibitors, demulsifiers, dehazers, antifoams, cetane number improvers, combustion improvers, antioxidants or stabilizers, antistats, metallocenes, metal deactivators, dyes and/or solvents.

In the case of gasoline fuels, these are primarily lubricity improvers (friction modifiers), corrosion inhibitors, demulsifiers, dehazers, antifoams, combustion improvers, antioxidants or stabilizers, antistats, metallocenes, metal deactivators, dyes and/or solvents.

Typical examples of suitable coadditives are listed in the following section:

### (1) Detergent Additives

Preferably, the customary detergent additives are amphiphilic substances which have at least one hydrophobic hydrocarbon radical with a number-averaged molecular weight (Mₙ) of 85 to 20 000 and at least one polar group which is selected from:
(a) mono- or polyamino groups having up to 6 nitrogen atoms, where at least one nitrogen atom has basic properties;
(b) nitro groups, optionally in combination with hydroxyl groups;
(c) hydroxyl groups in combination with mono- or polyamino groups, where at least one nitrogen atom has basic properties;
(d) carboxyl groups or alkali metal or alkaline earth metal salts thereof;
(e) sulfonic acid groups or alkali metal or alkaline earth metal salts thereof;
(f) polyoxy-C₂- to C₄-alkylene groups terminated by hydroxyl groups, mono- or polyamino groups, where at least one nitrogen atom has basic properties, or by carbamate groups;
(g) carboxylic acid ester groups;
(h) groups derived from succinic anhydride and having hydroxyl and/or amino and/or amido and/or imido groups; and/or
(i) groups generated by Mannich reaction of substituted phenols with aldehydes and mono- or polyamines.

The hydrophobic hydrocarbon radical in the above detergent additives, which ensures adequate solubility in the fuel, has a number-averaged molecular weight (Mₙ) of 85 to 20 000, preferably from 113 to 10 000, particularly preferably from 300 to 5000, more preferably from 300 to 3000, even more preferably from 500 to 2500 and in particular from 700 to 2500, especially from 800 to 1500. Typical hydrophobic hydrocarbon radicals are especially polypropenyl, polybutenyl and polyisobutenyl radicals with a number-averaged molecular weight Mₙ of preferably in each case 300 to 5000, particularly preferably 300 to 3000, more preferably 500 to 2500, even more preferably 700 to 2500 and in particular 800 to 1500.

Examples of the above groups of detergent additives which may be mentioned are the following:

additives comprising mono- or polyamino groups (a) are preferably polyalkene mono- or polyalkene polyamines based on polypropene or on high-reactivity (i.e. having predominantly terminal double bonds) or conventional (i.e. having predominantly internal double bonds) polybutene or polyisobutene with Mₙ=300 to 5000, particularly preferably 500 to 2500 and in particular 700 to 2500. Additives of this type based on high-reactivity polyisobutene, which can be prepared from the polyisobutene, which can comprise up to 20% by weight of n-butene units, by hydroformylation and reductive amination with ammonia, monoamines or polyamines such as dimethylaminopropylamine, ethylenediamine, diethylenetriamine, triethylenetetramine or tetraethylenepentamine, are known in particular from EP-A 244 616. When, in the preparation of the additives, polybutene or polyisobutene with predominantly internal double bonds (mostly in the β- and γ positions) are used as starting materials, the preparation route is by chlorination and subsequent amination or by oxidation of the double bond with air or ozone to give the carbonyl or carboxyl compound and subsequent amination under reductive (hydrogenating) conditions. Amines which can be used here for the amination are e.g. ammonia, monoamines or the aforementioned polyamines. Corresponding additives based on polypropene are described in particular in WO-A 94/24231.

Further particular additives comprising monoamino groups (a) are the hydrogenation products of the reaction products of polyisobutenes having an average degree of polymerization P=5 to 100 with nitrogen oxides or mixtures of nitrogen oxides and oxygen, as are described in particular in WO-A 97/03946.

Further particular additives comprising monoamino groups (a) are the compounds obtainable from polyisobutene epoxides by reaction with amines and subsequent dehydrogenation and reduction of the amino alcohols, as described in particular in DE-A 196 20 262.

Additives comprising nitro groups (b), optionally in combination with hydroxyl groups, are preferably reaction products of polyisobutenes of average degree of polymerization P=5 to 100 or 10 to 100 with nitrogen oxides or mixtures of nitrogen oxides and oxygen, as are described in particular in WO-A96/03367 and in WO-A 96/03479. These reaction products are generally mixtures of pure nitropolyisobutenes (e.g. α,β-dinitropolyisobutene) and mixed hydroxynitropolyisobutenes (e.g. α-nitro-β-hydroxypolyisobutene).

Additives comprising hydroxyl groups in combination with mono- or polyamino groups (c) are in particular reaction products of polyisobutene epoxides obtainable from polyisobutene having preferably predominantly terminal double bonds and Mₙ=300 to 5000, with ammonia, mono- or polyamines, as are described in particular in EP-A 476 485.

Additives comprising carboxyl groups or their alkali metal or alkaline earth metal salts (d) are preferably copolymers of C₂- to C₄₀-olefins with maleic anhydride with a total molar mass of 500 to 20 000 and some or all of whose carboxyl groups have been converted to the alkali metal or alkaline earth metal salts and any remaining carboxyl groups have been reacted with alcohols or amines. Such additives are known in particular from EP-A 307 815. Additives of this type serve primarily to prevent valve seat wear and can, as described in WO-A 87/01126, advantageously be used in combination with customary fuel detergents such as poly(iso)buteneamines or polyetheramines.

Additives comprising sulfonic acid groups or alkali metal or alkaline earth metal salts thereof (e) are preferably alkali metal or alkaline earth metal salts of a sulfosuccinic acid alkyl ester, as is described in particular in EP-A 639 632. Additives of this type serve primarily to prevent valve seat wear and can be used advantageously in combination with customary fuel detergents such as poly(iso)buteneamines or polyetheramines.

Additives comprising polyoxy-C₂-C₄-alkylene groups (f) are preferably polyethers or polyetheramines which are obtainable by reacting C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols with 1 to 30 mol of ethylene oxide and/or propylene oxide and/or butylene oxide per hydroxyl group or amino group and, in the case of the polyetheramines, by subsequent reductive amination with ammonia, monoamines or polyamines. Products of this type are described in particular in EP-A 310 875, EP-A 356 725, EP-A 700 985 and US-A 4 877 416. In the case of polyethers, such products also satisfy carrier oil properties. Typical examples thereof are tridecanol or isotridecanol butoxylates, isononylphenol butoxylates and polyisobutenol butoxylates and propoxylates and also the corresponding reaction products with ammonia.

Additives comprising carboxylic acid ester groups (g) are preferably esters of mono-, di- or tricarboxylic acids with long-chain alkanols or polyols, in particular those with a minimum viscosity of 2 mm²/s at 100° C., as are described in particular in DE-A 38 38 918. Mono-, di- or tricarboxylic acids which can be used are aliphatic or aromatic acids, and suitable ester alcohols or ester polyols are in particular long-chain representatives having, for example, 6 to 24 carbon atoms. Typical representatives of the esters are adipates, phthalates, isophthalates, terphthalates and trimellitates of isooctanol, isononanol, isodecanol and of isotridecanol. Products of this type also satisfy carrier oil properties.

Additives comprising groups derived from succinic anhydride and having hydroxyl and/or amino and/or amido and/or in particular imido groups (h) are preferably corresponding derivatives of alkyl- or alkenyl-substituted succinic anhydride and in particular the corresponding derivatives of polyisobutenylsuccinic anhydride, which are obtainable by reacting conventional or high-reactivity polyisobutene with Mₙ=preferably 300 to 5000, particularly preferably 300 to 3000, more preferably 500 to 2500, even more preferably 700 to 2500 and in particular 800 to 1500, with maleic anhydride by a thermal route in ene reaction or via the chlorinated polyisobutene. The groups having hydroxy and/or amino and/or amido and/or imido groups are, for example, carboxylic acid groups, acid amides of monoamines, acid amides of di- or polyamines which, besides the amide function, also have free amine groups, succinic acid derivatives with an acid and an amide function, carboximides with monoamines, carboximides with di- or polyamines which, besides the imide function, also have free amine groups, or diimides, which are formed by reacting di- or polyamines with two succinic acid derivatives. In the event of the presence of imido groups (h), the further detergent additive within the context of the present invention is, however, used only up to a maximum of 100% of the total weight of compounds with betaine structure. Fuel additives of this type are generally known and are described, for example, in documents (1) and (2). They are preferably the reaction products of alkyl- or alkenyl-substituted succinic acids or derivatives thereof with amines and particularly preferably the reaction products of polyisobutenyl-substituted succinic acids or derivatives thereof with amines. Of particular interest here are reaction products with aliphatic polyamines (polyalkyleneimines) such as, in particular, ethylenediamine, diethylenetriamine, triethylenetetramine, tetraethylenepentamine, pentaethylenehexamine and hexaethyleneheptamine, which have an imide structure.

Additives comprising groups (i) produced by Mannich reaction of substituted phenols with aldehydes and mono- or polyamines are preferably reaction products of polyisobutene-substituted phenols with formaldehyde and mono- or polyamines such as ethylenediamine, diiethylenetriamine, triethylenetetramine, tetraethylenepentamine or dimethylaminopropylamine. The polyisobutenyl-substituted phenols can stem from conventional or high-reactivity polyisobutene with Mₙ=300 to 5000. “Polyisobutene Mannich bases” of this kind are described in particular in EP-A 831 141.

One or more of the specified detergent additives can be added to the fuel in an amount such that the dosing rate of these detergent additives is preferably 25 to 2500 ppm by weight, in particular 75 to 1500 ppm by weight, especially 150 to 1000 ppm by weight.

### (2) Carrier Oils

Co-used carrier oils may be mineral or synthetic in nature. Suitable mineral carrier oils are fractions produced in the processing of crude oil, such as brightstock or base oils having viscosities such as, for example, from the class SN 500 to 2000, but also aromatic hydrocarbons, paraffinic hydrocarbons and alkoxyalkanols. It is likewise possible to use a fraction which is produced in the refining of mineral oil and is known as “hydrocrack oil” (vacuum distillate cut with a boiling range from about 360 to 500° C., obtainable from natural mineral oil which has been catalytically hydrogenated and isomerized under high pressure and also deparaffinized). Mixtures of the aforementioned mineral carrier oils are likewise suitable.

Examples of suitable synthetic carrier oils are polyolefins (polyalphaolefins or polyinternalolefins), (poly)ester, (poly)alkoxylates, polyethers, aliphatic polyetheramines, alkylphenol-started polyethers, alkylphenol-started polyetheramines and carboxylic acid esters of long-chain alkanols.

Examples of suitable polyolefins are olefin polymers having Mₙ=400 to 1800, in particular based on polybutene or polyisobutene (hydrogenated or unhydrogenated).

Examples of suitable polyethers or polyetheramines are preferably compounds comprising polyoxy-C₂- to C₄-alkylene groups which are obtainable by reacting C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols having 1 to 30 mol of ethylene oxide and/or propylene oxide and/or butylenes oxide per hydroxyl group or amino group and, in the case of the polyetheramines, by subsequent reductive amination with ammonia, monoamines or polyamines. Products of this type are described in particular in EP-A 310 875, EP-A 356 725, EP-A 700 985 and U.S. Pat. No. 4,877,416. For example, the polyetheramines used may be poly-C₂- to C₆-alkylene oxide amines or functional derivatives thereof. Typical examples thereof are tridecanol or isotridecanol butoxylates, isononylphenol butoxylates and polyisobutenol butoxylates and propoxylates, and also the corresponding reaction products with ammonia.

Examples of carboxylic acid esters of long-chain alkanols are in particular esters of mono-, di- or tricarboxylic acids with long-chain alkanols or polyols, as are described in particular in DE-A 38 38 918. Mono-, di- or tricarboxylic acids which can be used are aliphatic or aromatic acids, and suitable esters alcohols and ester polyols are in particular long-chain representatives having, for example, 6 to 24 carbon atoms. Typical representatives of the esters are adipates, phthalates, isophthalates, terephthalates and trimellitates of isooctanol, isononanol, isodecanol and isotridecanol, e.g. di(n- or isotridecyl)phthalate.

Further suitable carrier oil systems are described, for example, in DE-A 38 26 608, DE-A 41 42 241, DE-A 43 09 074, EP-A 452 328 and EP-A 548 617.

Examples of particularly suitable synthetic carrier oils are alcohol-started polyethers having about 5 to 35, preferably about 5 to 30, particularly preferably 10 to 30 and in particular 15 to 30, C₃- to C₆-alkylene oxide units, e.g. propylene oxide, n-butylene oxide and isobutylene oxide units or mixtures thereof, per alcohol molecule. Nonlimiting examples of suitable starter alcohols are long-chain alkanols or phenols substituted with long-chain alkyl, where the long-chain alkyl radical is in particular a straight-chain or branched C₆- to C₁₈-alkyl radical. Particular examples to be mentioned are tridecanol and nonylphenol. Particularly preferred alcohol-started polyethers are the reaction products (polyetherification products) of monohydric aliphatic C₆- to C₁₈-alcohols with C₃- to C₆-alkylene oxides. Examples of monohydric aliphatic C₆-C₁₈-alcohols are hexanol, heptanol, octanol, 2-ethylhexanol, nonyl alcohol, decanol, 3-propylheptanol, undecanol, dodecanol, tridecanol, tetradecanol, pentadecanol, hexadecanol, octadecanol and the constitutional and positional isomers thereof. The alcohols can be used either in the form of the pure isomers or in the form of technical-grade mixtures. A particularly preferred alcohol is tridecanol. Examples of C₃- to C₆-alkylene oxides are propylene oxide, such as 1,2-propylene oxide, butylene oxide, such as 1,2-butylene oxide, 2,3-butylene oxide, isobutylene oxide or tetrahydrofuran, pentylene oxide and hexylene oxide. Among these, particular preference is given to C₃- to C₄-alkylene oxides, i.e. propylene oxide such as 1,2-propylene oxide and butylene oxide such as 1,2-butylene oxide, 2,3-butylene oxide and isobutylene oxide. Specifically, butylene oxide is used.

Further suitable synthetic carrier oils are alkoxylated alkylphenols, as described in DE-A 10 102 913.

Particular carrier oils are synthetic carrier oils, particular preference being given to the alcohol-started polyethers described above.

The carrier oil or the mixture of different carrier oils is added to the fuel in an amount of preferably 1 to 1000 ppm by weight, particular preferably from 10 to 500 ppm by weight and in particular from 20 to 100 ppm by weight.

### (3) Cold Flow Improvers

Suitable cold flow improvers are in principle all organic compounds which are able to improve the flow behavior of medium distillate fuels or diesel fuels at low temperatures. They must expediently have adequate oil solubility. In particular, the cold flow improvers usually used in the case of middle distillates of fossil origin, i.e. in the case of customary mineral diesel fuels (“middle distillate flow improvers”), (“MDFIs”) are suitable for this purpose. However, it is also possible to use organic compounds which, when used in customary diesel fuels, partly or predominantly have the properties of a wax antisettling additive (“WASA”). They can also act partly or predominantly as nucleators. It is also possible, however, to use mixtures of organic compounds effective as MDFI and/or effective as WASA and/or effective as nucleators.

Typically, the cold flow improver is selected from:

(K1) copolymers of a C₂- to C₄₀-olefin with at least one further ethylenically unsaturated monomer;
(K2) comb polymers;
(K3) polyoxyalkylenes;
(K4) polar nitrogen compounds
(K5) sulfocarboxylic acids or sulfonic acids or derivatives thereof; and
(K6) poly(meth)acrylic acid esters.

It is possible to use either mixtures of different representatives from one of the particular classes (K1) to (K6) or mixtures of representatives from different classes (K1) to (K6).

Suitable C₂- to C₄₀-olefin monomers for the copolymers of class (K1) are, for example, those having 2 to 20, in particular 2 to 10 carbon atoms, and also having 1 to 3, preferably having 1 or 2, in particular having 1 carbon-carbon double bond. In the last-mentioned case, the carbon-carbon double bond can be arranged either terminally (α-olefins) or internally. However, preference is given to α-olefins, particularly preferably α-olefins having 2 to 6 carbon atoms, for example propene, 1-butene, 1-pentene, 1-hexene and in particular ethylene.

In the case of the copolymers of class (K1), the at least one further ethylenically unsaturated monomer is preferably selected from carboxylic acid alkenyl esters, (meth)acrylic acid esters and further olefins.

If further olefins are co-polymerized, these preferably have a higher molecular weight than the aforementioned C₂- to C₄₀-olefin base monomer. If, for example, the olefin base monomer used is ethylene or propene, suitable further olefins are in particular C₁₀- to C₄₀-α-olefins. Further olefins are in most cases only additionally copolymerized when monomers with carboxylic acid ester functions are also used.

Suitable (meth)acrylic acid esters are, for example, esters of (meth)acrylic acid with C₁- to C₂₀-alkanols, in particular C₁- to C₁₀-alkanols, in particular with methanol, ethanol, propanol, isopropanol, n-butanol, sec-butanol, isobutanol, tert-butanol, pentanol, hexanol, heptanol, octanol, 2-ethylhexanol, nonanol and decanol, and structural isomers thereof.

Suitable carboxylic acid alkenyl esters are, for example, C₂- to C₁₄-alkenyl esters, e.g. the vinyl and propenyl esters, of carboxylic acids having 2 to 21 carbon atoms, the hydrocarbon radical of which may be linear or branched. Among these, preference is given to the vinyl esters. Among the carboxylic acids with a branched hydrocarbon radical, preference is given to those whose branch is in the α position relative to the carboxyl group, in which case the α carbon atom is particularly preferably tertiary, i.e. the carboxylic acid is a so-called neocarboxylic acid. Preferably, however, the hydrocarbon radical of the carboxylic acid is linear.

Examples of suitable carboxylic acid alkenyl esters are vinyl acetate, vinyl propionate, vinyl butyrate, vinyl-2-ethylhexanoate, vinyl neopentanoate, vinyl hexanoate, vinyl neononanoate, vinyl neodecanoate and the corresponding propenyl esters, with the vinyl esters being preferred. A particularly preferred carboxylic acid alkenyl ester is vinyl acetate; typical copolymers of group (K1) resulting therefrom are the most often used ethylene-vinyl acetate copolymers (“EVAs”).

Ethylene-vinyl acetate copolymers which can be used particularly advantageously and their preparation are described in WO 99/29748.

Suitable copolymers of class (K1) are also those which comprise two or more different carboxylic acid alkenyl esters in copolymerized form, which differ in the alkenyl function and/or in the carboxylic acid group. Likewise of suitability are copolymers which, as well as the carboxylic acid alkenyl ester(s), comprise at least one olefin and/or at least one (meth)acrylic acid ester in copolymerized form.

Terpolymers of a C₂- to C₄₀-α olefin, a C₁- to C₂₀-alkylester of an ethylenically unsaturated monocarboxylic acid having 3 to 15 carbon atoms and a C₂- to C₁₄-alkenyl ester of a saturated monocarboxylic acid having 2 to 21 carbon atoms are also suitable as copolymers of class (K1). Terpolymers of this type are described in WO 2005/054314. A typical terpolymer of this type is composed of ethylene, 2-ethylhexyl acrylate and vinyl acetate.

The at least one or the further ethylenically unsaturated monomer(s) are copolymerized in the copolymers of class (K1) in an amount of preferably 1 to 50% by weight, in particular from 10 to 45% by weight and in particular from 20 to 40% by weight, based on the total copolymer. The main fraction, in terms of weight, of the monomer units in the copolymers of class (K1) therefore usually originates from the C₂ to C₄₀ base olefins.

The copolymers of class (K1) preferably have a number-average molecular weight Mₙ of from 1000 to 20 000, particularly preferably from 1000 to 10 000 and in particular from 1000 to 8000.

Typical comb polymers of component (K2) are, for example, obtainable by the copolymerization of maleic anhydride or fumaric acid with another ethylenically unsaturated monomer, for example with an α-olefin or an unsaturated ester such as vinyl acetate, and subsequent esterification of the anhydride or acid function with an alcohol having at least 10 carbon atoms. Further suitable comb polymers are copolymers of α-olefins and esterified comonomers, for example esterified copolymers of styrene and maleic anhydride or esterified copolymers of styrene and fumaric acid.

Suitable comb polymers may also be polyfumarates or polymaleates. Moreover, homo- and copolymers of vinyl ethers are suitable comb polymers. Comb polymers suitable as component of class (K2) are, for example, also those described in WO 2004/035715 and in “Comb-Like Polymers. Structure and Properties, N. A. Platé and V. P. Shibaev, J. Poly. Sci. Macromolecular Revs. 8, pages 117 to 253 (1974)”. Mixtures of comb polymers are also suitable.

Polyoxyalkylenes suitable as component of class (K3) are, for example, polyoxyalkylene esters, polyoxyalkylene ethers, mixed polyoxyalkylene ester ethers and mixtures thereof. These polyoxyalkylene compounds preferably comprise at least one, preferably at least two linear alkyl groups having in each case 10 to 30 carbon atoms and a polyoxyalkylene group with a number-average molecular weight of up to 5000. Polyoxyalkylene compounds of this type are described, for example, in EP-A 061 895 and also in U.S. Pat. No. 4,491,455. Particular polyoxyalkylene compounds are based on polyethylene glycols and polypropylene glycols with a number-average molecular weight of from 100 to 5000. Furthermore, polyalkylene mono- and diesters of fatty acids having 10 to 30 carbon atoms, such as stearic acid or behenic acid, are also suitable.

Polar nitrogen compounds suitable as component of class (K4) may either be ionic or nonionic in nature and preferably have at least one, in particular at least two substituents in the form of a tertiary nitrogen atom of the general formula >NR⁷, in which R⁷ is a C₈- to C₄₀-hydrocarbon radical. The nitrogen substituents can also be present in quaternized form, i.e. in cationic form. Examples of such nitrogen compounds are ammonium salts and/or amides which are obtainable by the reaction of at least one amine substituted with at least one hydrocarbon radical with a carboxylic acid having 1 to 4 carboxyl groups or with a suitable derivative thereof. Preferably, the amines comprise at least one linear C₈- to C₄₀-alkyl radical. Primary amines suitable for preparing the specified polar nitrogen compounds are, for example, octylamine, nonylamine, decylamine, undecylamine, dodecylamine, tetradecylamine and the higher linear homologs; secondary amines suitable for this purpose are, for example, dioctadecylamine and methylbehenylamine. Of suitability for this purpose are also amine mixtures, in particular amine mixtures obtainable on an industrial scale such as fatty amines or hydrogenated tallamines, as are described, for example, in Ullmanns Encyclopedia of Industrial Chemistry, 6^{th} edition, in the chapter “Amines, aliphatic”. Acids suitable for the reaction are, for example, cyclohexane-1,2-dicarboxylic acid, cyclohexene-1,2-dicarboxylic acid, cyclopentane-1,2-dicarboxylic acid, naphthalenedicarboxylic acid, phthalic acid, isophthalic acid, terephthalic acid and succinic acids substituted with long-chain hydrocarbon radicals.

In particular, the component of class (K4) is an oil-soluble reaction product of poly(C₂- to C₂₀-carboxylic acids) having at least one tertiary amino group with primary or secondary amines. The poly(C₂- to C₂₀-carboxylic acids) which have at least one tertiary amino group and form the basis of this reaction product comprise preferably at least 3 carboxyl groups, in particular 3 to 12, especially 3 to 5 carboxyl groups. The carboxylic acid units in the polycarboxylic acids preferably have 2 to 10 carbon atoms, and are in particular acetic acid units. The carboxylic acid units are suitably linked to the polycarboxylic acids, mostly via one or more carbon and/or nitrogen atoms. They are preferably bonded to tertiary nitrogen atoms which, in the case of a plurality of nitrogen atoms, are bonded via hydrocarbon chains.

Preferably, the component of class (K4) is an oil-soluble reaction product based on poly(C₂- to C₂₀-carboxylic acids) which have at least one tertiary amino group and are of the general formula IIa or IIb

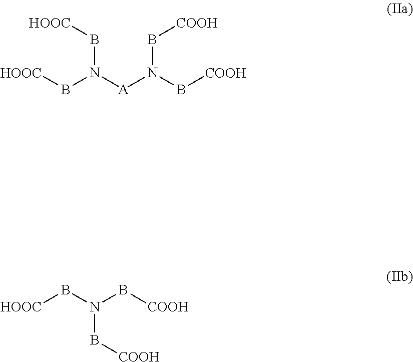

in which the variable A is a straight-chain or branched C₂- to C₆-alkylene group or the group of the formula III

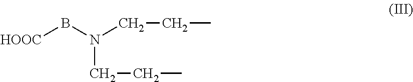

and the variable B is a C₁- to C₁₉-alkylene group. The compounds of the general formula IIa and IIb have in particular the properties of a WASA.

Furthermore, the preferred oil-soluble reaction product of component (K4), in particular that of the general formula IIa or IIb, is an amide, an amide-ammonium salt or an ammonium salt in which no, one or more carboxylic acid groups have been converted to amide groups.

Straight-chain or branched C₂- to C₆-alkylene groups of the variables A are, for example, 1,1-ethylene, 1,2-propylene, 1,3-propylene, 1,2-butylene, 1,3-butylene, 1,4-butylene, 2-methyl-1,3-propylene, 1,5-pentylene, 2-methyl-1,4-butylene, 2,2-dimethyl-1,3-propylene, 1,6-hexylene (hexamethylene) and in particular 1,2-ethylene. The variable A preferably comprises 2 to 4, in particular 2 or 3 carbon atoms.

C₁- to C₁₉-alkylene groups of the variables B are, for example, 1,2-ethylene, 1,3-propylene, 1,4-butylene, hexamethylene, octamethylene, decamethylene, dodecamethylene, tetradecamethylene, hexadecamethylene, octadecamethylene, nonadecamethylene and in particular methylene. The variable B preferably comprises 1 to 10, in particular 1 to 4, carbon atoms.

The primary and secondary amines as a reaction partner for the polycarboxylic acids to form the component (K4) are usually monoamines, in particular aliphatic monoamines. These primary and secondary amines can be selected from a large number of amines which carry hydrocarbon radicals which are optionally bonded to one another.

In most cases, these amines which form the basis of the oil-soluble reaction products of component (K4) are secondary amines and have the general formula HN(R⁸)₂, in which the two variables R⁸ independently of one another, are in each case straight-chain or branched C₁₀- to C₃₀-alkyl radicals, in particular C₁₄- to C₂₄-alkyl radicals. These relatively long-chain alkyl radicals are preferably straight-chain or only slightly branched. In general, the specified secondary amines, with regard to their relatively long-chain alkyl radicals, are derived from naturally occurring fatty acid and from derivatives thereof. The two radicals R⁸ are preferably identical.

The specified secondary amines can be bonded to the polycarboxylic acids by means of amide structures or in the form of the ammonium salts; it is also possible for only some to be present as amide structures and others to be present as ammonium salts. Preferably, only few, if any, free acid groups are present. The oil-soluble reaction products of component (K4) are preferably present completely in the form of the amide structures.

Typical examples of such components (K4) are reaction products of nitrilotriacetic acid, of ethylenediaminetetraacetic acid or of propylene-1,2-diaminetetraacetic acid having in each case 0.5 to 1.5 mol per carboxyl group, in particular 0.8 to 1.2 mol per carboxyl group, dioleylamine, dipalmitinamine, dicoconut fatty amine, distearylamine, dibehenylamine or in particular ditallow fatty amine. A particularly preferred component (K4) is the reaction product of 1 mol of ethylenediaminetetraacetic acid and 4 mol of hydrogenated ditallow fatty amine.

Further typical examples of component (K4) which may be mentioned are the N,N-dialkylammonium salts of 2-N′,N-dialkylamidobenzoates, for example the reaction product of 1 mol of phthalic anhydride and 2 mol of ditallow fatty amine, where the latter may be hydrogenated or unhydrogenated, and the reaction product of 1 mol of an alkenylspirobislactone with 2 mol of a dialkylamine, for example ditallow fatty amine and/or tallow fatty amine, where the last two may be hydrogenated or unhydrogenated.

Further typical structure types for the component of class (K4) are cyclic compounds with tertiary amino groups or condensates of long-chain primary or secondary amines with carboxylic acid-containing polymers, as described in WO 93/18115.

Sulfocarboxylic acids, sulfonic acids or derivatives thereof which are suitable as cold flow improvers of the component of class (K5) are, for example, the oil-soluble carboxamides and carboxylic acid esters of ortho-sulfobenzoic acid in which the sulfonic acid function is present as sulfonate with alkyl-substituted ammonium cations, as are described in EP-A 261 957.

Poly(meth)acrylic acid esters suitable as cold flow improvers of the component of class (K6) are either homo- or copolymers of acrylic acid and methacrylic acid esters. Preference is given to copolymers of at least two different (meth)acrylic acid esters which differ with regard to the condensed-in alcohol. The copolymer optionally comprises another different olefinically unsaturated monomer in copolymerized form. The weight-average molecular weight of the polymer is preferably 50 000 to 500 000. A particularly preferred polymer is a copolymer of methacrylic acid and methacrylic acid esters of saturated C₁₄- and C₁₅-alcohols, where the acid groups have been neutralized with hydrogenated tallamine. Suitable poly(meth)acrylic acid esters are described, for example, in WO 00/44857.

The cold flow improver or the mixture of different cold flow improvers is added to the middle distillate fuel or diesel fuel in a total amount of preferably 10 to 5000 ppm by weight, particularly preferably of 20 to 2000 ppm by weight, more preferably of 50 to 1000 ppm by weight and in particular of 100 to 700 ppm by weight, e.g. 200 to 500 ppm by weight.

### (4) Lubricity Improvers

Suitable lubricity improvers (or friction modifiers) are usually based on fatty acids or fatty acid esters. Typical examples are tall oil fatty acid, as described, for example, in WO 98/004656, and glycerol monooleate. The reaction products, described in U.S. Pat. No. 6,743,266 B2, of natural or synthetic oils, for example triglycerides, and alkanolamines are also suitable as such lubricity improvers.

### (5) Corrosion Inhibitors

Suitable corrosion inhibitors are e.g. succinic acid esters, especially with polyols, fatty acid derivatives, e.g. oleic acid esters, oligomerized fatty acids, substituted ethanolamines and products which are sold under the trade name RC 4801 (Rhein Chemie Mannheim, Germany) or HiTEC 536 (Ethyl Corporation).

### (6) Demulsifiers

Suitable demulsifiers are e.g. the alkali metal or alkaline earth metal salts of alkyl-substituted phenol- and naphthalenesulfonates and the alkali metal or alkaline earth metal salts of fatty acids, moreover neutral compounds such as alcohol alkoxylates, e.g. alcohol ethoxylates, phenol alkoxylates, e.g. tert-butylphenol ethoxylate or tert-butylphenol ethoxylate, fatty acids, alkylphenols, condensation products of ethylene oxide (EO) and propylene oxide (PO), e.g. including in the form of EO/PO block copolymers, polyethyleneimines or else polysiloxanes.

### (7) Dehazers

Suitable dehazers are e.g. alkoxylated phenol-formaldehyde condensates, such as, for example, the products available under the trade name NALCO 7D07 (Nalco) and TOLAD 2683 (Petrolite).

### (8) Antifoams

Suitable antifoams are e.g. polyether-modified polysiloxanes, such as, for example, the products available under the trade name TEGOPREN 5851 (Goldschmidt), Q 25907 (Dow Corning) and RHODOSIL (Rhone Poulenc).

### (9) Cetane Number Improvers

Suitable cetane number improvers are e.g. aliphatic nitrates such as 2-ethylhexyl nitrate and cyclohexyl nitrate, and also peroxides such as di-tert-butyl peroxide.

### (10) Antioxidants

Suitable antioxidants are e.g. substituted phenols, such as 2,6-di-tert-butylphenol and 6-di-tert-butyl-3-methylphenol, and also phenylenediamine such as N,N′-di-sec-butyl-p-phenylenediamine.

### (11) Metal Deactivators

Suitable metal deactivators are e.g. salicylic acid derivatives such as N,N′-disalicylidene-1,2-propanediamine.

### (12) Solvents

Suitable solvents are e.g. nonpolar organic solvents such as aromatic and aliphatic hydrocarbons, for example toluene, xylenes, “white spirit” and products which are sold under the trade name SHELLSOL (Royal Dutch/Shell Group) and EXXSOL (ExxonMobil), and also polar organic solvents, for example alcohols such as 2-ethylhexanol, decanol and isotridecanol. Solvents of this type are in most cases used in the diesel fuel together with the aforementioned additives and coadditives, which they are intended to dissolve or dilute for the purposes of better handling.

### D5) Solubilizers

According to the invention, the aforementioned farnesyl alkoxylates according to the invention can also be as solubilizers for aqueous systems in order to solubilize water-insoluble substances therein.

In this connection, the compounds according to the invention are usually used in amounts of 0.01 to 50% by weight or 0.1 to 10% by weight or 1 to 8% by weight, based on the total weight of the solubilized mixture. Optionally, in addition, further surface-active additives such as e.g. selected from the surfactants described above, may be present.

### D6) Agrochemical Compositions

The compounds according to the invention of the formula I are also suitable for use as adjuvants in agrochemical compositions or crop protection formulations, e.g. in herbicidal, fungicidal or insecticidal compositions.

The compounds I can be converted to the types customary for agrochemical compositions, e.g. solutions, emulsions, suspensions, dusts, powders, pastes and granules. The type of composition is determined by the particular intended use.

The compositions comprise generally 0.01 to 20% by weight, 0.1 to 10% by weight or 1 to 5% by weight of the compounds I.

The agrochemical compositions are produced in a known manner (see e.g. U.S. Pat. No. 3,060,084, EP-A 707 445 (for liquid concentrates), Browning “Agglomeration”, Chemical Engineering, Dec. 4, 1967, 147-48, Perry's Chemical Engineer's Handbook, 4th edition, McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation technology (Wiley VCH Verlag, Weinheim, 2001).

Besides the active ingredient or the active ingredients and the compounds according to the invention of the formula I, the agrochemical compositions can furthermore also comprise other auxiliaries customary for crop protection compositions, the selection of the auxiliaries being determined according to the specific application form and/or the active ingredient.

Examples of suitable auxiliaries are solvents, solid carriers, surface-active substances (such as further solubilizers, protective colloids, wetting agents and adhesives), organic and inorganic thickeners, bactericides, antifreezes, antifoams, optionally dyes and stickers (e.g. for the treatment of seed material).

Suitable solvents are water, organic solvents such as mineral oil fractions of medium to high boiling point, such as kerosene and diesel oil, also coal tar oils, and oils of vegetable or animal origin, aliphatic, cyclic and aromatic hydrocarbons, e.g. paraffins, tetrahydronaphthalene, alkylated naphthalenes and derivatives thereof, alkylated benzenes and derivatives thereof, alcohols such as methanol, ethanol, propanol, butanol and cyclohexanol, glycols, ketones such as cyclohexanone, gamma-butyrolactone, dimethyl fatty acid amines, fatty acids and fatty acid esters and strongly polar solvents, e.g. amines such as N-methylpyrrolidone. In principle, it is also possible to use solvent mixtures and also mixtures of the aforementioned solvents and water.

Solid carriers are mineral earths such as silicas, silica gels, silicates, talc, kaolin, limestone, lime, chalk, bolus, loess, clay, dolomite, diatomaceous earth, calcium sulfate and magnesium sulfate, magnesium oxide, ground plastics, fertilizers, such as ammonium sulfate, ammonium phosphate, ammonium nitrate, ureas and vegetable products such as cereal flour, bark flour, wood flour and nut shell flour, cellulose powder or other solid carriers.

Suitable surface-active substances (adjuvants, wetting agents, adhesives, dispersants or emulsifiers) that are different from compounds of the formula I are the alkali metal, alkaline earth metal, ammonium salts of aromatic sulfonic acids, e.g. of lignin (Borresperse® grades, Borregaard, Norway), phenol, naphthalene (Morwet® grades, Akzo Nobel, USA) and dibutylnaphthalenesulfonic acid (Nekal® grades, BASF, Germany), and also of fatty acids, alkyl- and alkylarylsulfonates, alkyl sulfates, lauryl ether sulfates and fatty alcohol sulfates, and also salts of sulfated hexa-, hepta- and octadecanols, and also of fatty alcohol glycol ethers, condensation products of sulfonated naphthalene and its derivatives with formaldehyde, condensation products of naphthalene or of naphthalenesulfonic acids with phenol and formaldehyde, polyoxyethylene octylphenol ether, ethoxylated isooctyl-, octyl- or nonylphenol, alkylphenyl, tributylphenyl polyglycol ethers, alkylaryl polyether alkohols, isotridecyl alcohol, fatty alcohol ethylene oxide condensates, ethoxylated castor oil, polyoxyethylene or polyoxypropylene alkyl ethers, lauryl alcohol polyglycol ether acetate, sorbitol esters, ligninosulfite spent liquors, and proteins, denatured proteins, polysaccharides (e.g. methylcellulose), hydrophobically modified starches, polyvinyl alcohol (Mowiol® grades, Clariant, Switzerland), polycarboxylates (Sokalan® grades, BASF, Germany), polyalkoxylates, polyvinylamine (Lupamin® grades, BASF, Germany), polyethyleneimine (Lupasol® grades, BASF, Germany), polyvinylpyrrolidone and copolymers thereof.

Examples of thickeners (i.e. compounds which impart a modified flow behavior to the formulation, i.e. high viscosity in the resting state and low viscosity in the agitated state) are polysaccharides, and organic and inorganic layered minerals such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added to stabilize the composition. Examples of bactericides are those based on dichlorophene and benzyl alcohol hemiformal (Proxel® from ICI or Acticide® RS from Thor Chemie and Kathon® MK from Rohm & Haas), and isothiazolinone derivatives such as alkylisothiazolinones and benzisothiazolinones (Acticide® MBS from Thor Chemie).

Examples of suitable antifreezes are ethylene glycol, propylene glycol, urea and glycerol.

Examples of antifoams are silicone emulsions (such as e.g. Silikon® SRE, Wacker, Germany or Rhodorsil®, Rhodia, France), long-chain alcohols, fatty acids, salts of fatty acids, organofluorine compounds and mixtures thereof.

Examples of colorants are those sparingly water-soluble pigments and also water-soluble dyes. Examples which may be mentioned are the dyes and pigments known under the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of stickers are polyvinylpyrrolidone, polyvinyl acetate, polyvinyl alcohol and cellulose ether (Tylose®, Shin-Etsu, Japan).

Of suitability for producing directly sprayable solutions, emulsions, pastes or oil dispersions are mineral oil fractions of medium to high boiling point, such as kerosene or diesel oil, also cold tar oils and also oils of vegetable or animal origin, aliphatic, cyclic and aromatic hydrocarbons, e.g. toluene, xylene, paraffin, tetrahydronaphthalene, alkylated naphthalenes or derivatives thereof, methanol, ethanol, propanol, butanol, cyclohexanol, cyclohexanone, isophorone, strongly polar solvents, e.g. dimethyl sulfoxide, N-methylpyrrolidone or water.

Powders, grits and dusts can be produced by mixing or joint grinding of the compounds I and also, if present, further active ingredients with at least one solid carrier.

Granules, e.g. coated granules, impregnation granules and homogeneous granules, can be produced by binding the active ingredients to at least one solid carrier. Solid carriers are e.g. mineral earths, such as silica gels, silicates, talc, kaolin, attaclay, limestone, lime, chalk, bolus, loess, clay, dolomite, diatomaceous earth, calcium sulfate and magnesium sulfate, magnesium oxide, ground plastics, fertilizers such as ammonium sulfate, ammonium phosphate, ammonium nitrate, ureas and vegetable products such as cereal flour, bark flour, wood flour and nut shell flour, cellulose powder and other solid carriers.

Oils of various types, wetting agents, adjuvants, herbicides, bactericides, other fungicides and/or pest control agents, can be added to the active ingredients or to the compositions comprising these, optionally also only directly prior to application (tank mix). These agents can be added to the compositions according to the invention in the weight ratio 1:100 to 100:1, preferably 1:10 to 10:1.

Suitable adjuvants in this connection are in particular: organically modified polysiloxanes, e.g. Break Thru S 240®; alcohol alkoxylates, e.g. Atplus® 245, Atplus® MBA 1303, Plurafac® LF 300 and Lutensol® ON 30; EO-PO block polymers, e.g. Pluronic® RPE 2035 and Genapol® B; alcohol ethoxylates, e.g. Lutensol® XP 80; and sodium dioctylsulfosuccinate, e.g. Leophen® RA.

The compositions according to the invention can comprise one or more active ingredients selected from herbicides, insecticides, growth regulators, fungicides.

The following nonlimiting list names active ingredients with which the compounds according to the invention can be used together,

### A) Strobilurins:

azoxystrobin, dimoxystrobin, coumoxystrobin, coumethoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, methyl 2-[2-(2,5-dimethylphenyloxymethyl)phenyl)-3-methoxyacrylate, 2-(2-(3-(2,6-di-chlorophenyl)-1-methylallylideneaminooxymethyl)phenyl)-2-methoxyimino-N-methylacetamide;

### B) Carboxamides:

carboxanilides: benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methylthiazole-5-carboxanilide, N-(4′-trifluoromethylthiobiphenyl-2-yl)-3-difluoromethyl-1-methyl-1H-pyrazole-4-carboxamide, N-(2-(1,3,3-trimethyl-butyl)phenyl)-1,3-dimethyl-5-fluoro-1H-pyrazole-4-carboxamide;
carboxylic acid morpholides: dimethomorph, flumorph, pyrimorph;
benzamides: flumetover, fluopicolide, fluopyram, zoxamide;
other carboxamides: carpropamid, diclocymet, mandipropamid, oxytetracyclin, silthiofam, N-(6-methoxypyridin-3-yl)cyclopropanecarboxamide;

### C) Azoles:

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole;
imidazoles: cyazofamid, imazalil, imazalil sulfate, pefurazoate, prochloraz, triflumizole;
benzimidazoles: benomyl, carbendazim, fuberidazole, thiabendazole;
others: ethaboxam, etridiazole, hymexazole, 2-(4-chlorophenyl)-N-[4-(3,4-dimethoxyphenyl)isoxazol-5-yl]-2-prop-2-ynyloxyacetamide;

### D) Nitrogenous Heterocyclyl Compounds

pyridines: fluazinam, pyrifenox, 3-[5-(4-chlorophenyl)-2,3-dimethylisoxazolidin-3-yl]pyridine, 3-[5-(4-methylphenyl)-2,3-dimethylisoxazolidin-3-yl]pyridine;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph acetate, fenpropimorph, tridemorph;
piperidines: fenpropidin;
dicarboximides: fluoroimide, iprodione, procymidone, vinclozolin;
nonaromatic 5-membered heterocycles: famoxadone, fenamidone, flutianil, octhilinone, probenazole, S-allyl 5-amino-2-isopropyl-3-oxo-4-ortho-tolyl-2,3-dihydropyrazole-1-thiocarboxylate;
others: acibenzolar-5-methyl, amisulbrom, anilazine, blasticidin-S, captafol, captan, chinomethionat, dazomet, debacarb, diclomezine, difenzoquat, difenzoquat-methylsulfate, fenoxanil, folpet, oxolinic acid, piperalin, proquinazid, pyroquilon, quinoxyfen, triazoxide, tricyclazol, 2-butoxy-6-iodo-3-propylchromen-4-one, 5-chloro-1-(4,6-dimethoxypyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine, 5-ethyl-6-octyl-[1,2,4]triazolo[1,5-a]pyrimidin-7-ylamine;

### E) Carbamates and Dithiocarbamates

thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, 4-fluorophenyl N-(1-(1-(4-cyanophenyl)ethanesulfonyl)but-2-yl)carbamate;

### F) Other Fungicides

guanidines: dodine, dodine free base, guazatine, guazatine acetate, iminoctadine, iminoctadine triacetate, iminoctadine tris(albesilate);
antibiotics: kasugamycin, kasugamycin hydrochloride hydrate, polyoxins, streptomycin, validamycin A;
nitrophenyl derivatives: binapacryl, dicloran, dinobuton, dinocap, nitrothal isopropyl, tecnazene;
organometallic compounds: fentin salts, such as, for example, fentin acetate, fentin chloride, fentin hydroxide;
sulfur-comprising heterocyclyl compounds: dithianon, isoprothiolane;
organophosphorus compounds: edifenphos, fosetyl, fosetyl aluminum, iprobenfos, phosphorous acid and its salts, pyrazophos, tolclofos-methyl;
organochlorine compounds: chlorothalonil, dichlofluanid, dichlorophen, flusulfamide, hexachlorobenzene, pencycuron, pentachlorophenol and its salts, phthalide, quintozene, thiophanate methyl, tolylfluanid, N-(4-chloro-2-nitrophenyl)-N-ethyl-4-methylbenzenesulfonamide;
inorganic active ingredients: phosphorous acid and its salts, Bordeaux mixture, copper salts, such as, for example, copper acetate, copper hydroxide, copper oxychloride, basic copper sulfate, sulfur;
biological compositions for controlling fungi, plant strengthening products: Ampelomyces quisqualis (e.g. the product AQ 10® from Intrachem Bio GmbH & Co. KG, Germany), Aspergillus flavus (e.g. the product AFLAGUARD® from Syngenta, Switzerland), Aureobasidium pullulans (e.g. the product BOTECTOR® from bio-ferm GmbH, Germany), Bacillus pumllius (e.g. strain NRRL No. B-21661 in the products RHAPSODY®, SERENADE® MAX and SERENADE® ASO from AgraQuest Inc., USA), Bacillus subtilis var. amyloliquefaciens FZB24 (e.g. the product TAEGRO® from Novozyme Biologicals, Inc., USA), Candida oleophila I-82 (e.g. the product ASPIRE® from Ecogen Inc., USA), Candida saitoana (e.g. the products BIOCURE® (in a mixture with Lysozym) and BIOCOAT® from Micro Flo Company, USA (BASF SE) and Arysta), chitosan (e.g. ARMOUR-ZEN from Botri-Zen Ltd., New Zealand), Clonostachys rosea f. catenulata, also called Gliocladium catenulatum (e.g. strain J1446: PRESTOP® from Verdera, Finland), Coniothyrium minitans (e.g. the product CONTANS® from Prophyta, Germany), Cryphonectria parasitica (e.g. the product Endothia parasitica from CNICM, France), Cryptococcus albidus (e.g. the product YIELD PLUS® from Anchor BioTechnologies, South Africa), Fusarium oxysporum (e.g. the product BIOFOX® from S.I.A.P.A., Italy, and FUSACLEAN® from Natural Plant Protection, France), Metschnikowia fructicola (e.g. the product SHEMER® from Agrogreen, Israel), Microdochium dimerum (e.g. the product ANTIBOT® from Agrauxine, France), Phlebiopsis gigantea (e.g. the product ROTSOP® from Verdera, Finland), Pseudozyma flocculosa (e.g. the product SPORODEX® from Plant Products Co. Ltd., Canada), Pythium oligandrum DV74 (e.g. the product POLYVERSUM® from Remeslo SSRO, Biopreparaty, Czech Republic), Reynoutria sachlinensis (e.g. the product REGALIA® from Marrone Biolnnovations, USA), Talaromyces flavus V117b (e.g. the product PROTUS® from Prophyta, Germany), Trichoderma asperellum SKT-1 (e.g. the product ECO-HOPE® from Kumiai Chemical Industry Co., Ltd., Japan), T. atroviride LC52 (e.g. the product SENTINEL® from Agrimm Technologies Ltd, New Zealand), T. harzianum T-22 (e.g. the product PLANTSHIELD® from BioWorks Inc., USA), T. harzianum TH 35 (e.g. the product ROOT PRO® from Mycontrol Ltd., Israel), T. harzianum T-39 (e.g. the products TRICHODEX® and TRICHODERMA 2000® from Mycontrol Ltd., Israel and Makhteshim Ltd., Israel), T. harzianum and T. viride (e.g. the product TRICHOPEL from Agrimm Technologies Ltd, New Zealand), T. harzianum ICC012 and T. viride ICC080 (e.g. the product REMEDIER® WP from Isagro Ricerca, Italy), T. polysporum and T. harzianum (e.g. the product BINAB® from BINAB Bio-Innovation AB, Sweden), T. stromaticum (e.g. the product TRICOVAB® from C.E.P.L.A.C., Brazil), T. virens GL-21 (e.g. the product SOILGARD® from Certis LLC, USA), T. viride (e.g. the products TRIECO® from Ecosense Labs. (India) Pvt. Ltd., India and BIO-CURE® F from T. Stanes & Co. Ltd., India), T. viride TV1 (e.g. the product T. viride TV1 from Agribiotec srl, Italy), Ulocladium oudemansii HRU3 (e.g. the product BOTRY-ZEN® from Botry-Zen Ltd, New Zealand);
others: biphenyl, bronopol, cyflufenamid, cymoxanil, diphenylamine, metrafenone, pyriofenone, mildiomycin, oxine-copper, prohexadione-calcium, spiroxamine, tolylfluanid, N-(cyclopropylmethoxyimino-(6-difluoromethoxy-2,3-difluoro-phenyl)methyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethyl-silanylpropoxy)phenyl)-N-ethyl-N-methylformamidine, methyl N-(1,2,3,4-tetra-hydronaphthalen-1-yl)-2-{1-[2-(5-methyl-3-trifluoromethylpyrazol-1-yl)acetyl]piperidin-4-yl}thiazole-4-carboxamide, methyl (R)—N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-yl methoxyacetate, N-methyl-2-{1-[2-(5-methyl-3-trifluoromethyl-1H-pyrazol-1-yl)acetyl]piperidin-4-yl}-N-[(1R)-1,2,3,4-tetrahydro-naphthalen-1-yl]-4-thiazolecarboxamide;

### G) Growth Regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapac-ethyl and uniconazole;
   H) herbicides
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-p-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazone, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;
   I) insecticides
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
inhibitors of insect growth: a) chitin synthesis inhibitors: benzoylureas: chlorfluazuron, cyramazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]-triazinane;
GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorylation: cyhexatin, diafenthiuron, fenbutatin oxide, propargite;
insect molting inhibitors: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamid, chlorantraniliprol, cyazypyr (HGW86); cyenopyrafen, flupyrazofos, cyflumetofen, amidoflumet, imicyafos, bistrifluoron, and pyrifluquinazon.

The compositions serve e.g. to control a large number of pathogens on a variety of crop plants such as cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currants or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw material plants, for example corn, soybeans, wheat, oilseed rape, sugar cane or oil palms; corn; tobacco; nuts; coffee; tea; bananas; grapevines (grapes for eating and grapes for wine making); hops; grass, for example lawns; sweetleaf (Stevia rebaudania); rubber plants; ornamental and forest plants, for example flowers, shrubs, deciduous trees and coniferous trees, and also on the propagation material, for example seeds, and on the harvested material of these plants.

Moreover, the compositions according to the invention are suitable for controlling harmful fungi in the protection of stored products (and of harvested products) and in the protection of materials and buildings. The term “protection of materials and buildings” comprises the protection of industrial and non-living materials such as, for example, adhesives, glues, wood, paper and cardboard, textiles, leather, paint dispersions, plastic, cooling lubricants, fibers and tissues, against attack and destruction by unwanted microorganisms such as fungi and bacteria. In the protection of wood and materials, particular attention is paid to the following harmful fungi: Ascomycetes, such as Ophiostoma spp., Ceratocystis spp., Aureobasidium pullulans, Scierophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of materials to the following yeast fungi: Candida spp. and Saccharomyces cerevisae.

### Experimental Section

### A) Materials and Methods

Unless specified otherwise, the chemicals used are commercially available products.

Hydrogenated farnesol can be prepared in a manner known per. E.g. to prepare hexahydrofarnesol Raney-Ni (1% by weight based on farnesol) can be used under the following conditions: farnesol:ethanol 2:1 (mass ratio), 100° C., 70 bar, 15 h. The degree of hydrogenation can be adjusted by adjusting the molar ratio of farnesol to injected hydrogen.

### B) Preparation Examples

### Preparation Example 1

### Preparation of Hexahydrofarnesol-3 EO

A stirred reactor was charged with 327.1 g of hexahydrofarnesol (CAS No. 6750-34-1) and 1.26 g of a 44% strength aqueous KOH solution. The mixture was dewatered for 30 minutes at 120° C. and 20 mbar. It was then rendered inert with nitrogen up to a pressure of 0.5 bar. At a temperature of 160° C. and a pressure of at most 2.3 bar, 189.5 g of ethylene oxide were metered in. When the metered addition was complete, the reactor contents, cooled to 60° C., were neutralized by adding 0.66 g of acetic acid and the reactor was emptied.

Analysis

OH number=151 mg KOH/g

pH (5% strength aqueous solution): 5.6

Cloud point (method E in accordance with DIN EN 1890):43.7° C.

### Preparation Example 2

### Preparation of Hexahydrofarnesol-5 EO

A stirred reactor was charged with 263.2 g of hexahydrofarnesol and 1.26 g of a 44% strength aqueous KOH solution. The mixture was dewatered for 30 minutes at 120° C. and 20 mbar. It was then rendered inert with nitrogen up to a pressure of 0.5 bar. At a temperature of 160° C. and a pressure of at most 2.6 bar, 254.4 g of ethylene oxide were metered in. When the metered addition was complete, the reactor contents, cooled to 60° C., were neutralized by adding 0.64 g of acetic acid and the reactor was emptied.

Analysis

OH number=121.2 mg KOH/g

pH (5% strength aqueous solution): 5.6

Cloud point (method E in accordance with DIN EN 1890): 64.9° C.

### Preparation Example 3

### Preparation of Hexahydrofarnesol-7 EO

A stirred reactor was charged with 220 g of hexahydrofarnesol and 1.32 g of a 44% strength aqueous KOH solution. The mixture was dewatered for 30 minutes at 120° C. and 20 mbar. It was then rendered inert with nitrogen up to a pressure of 0.5 bar. At a temperature of 160° C. and a pressure of at most 2.8 bar, 297.6 g of ethylene oxide were metered in. When the metered addition was complete, the reactor contents, cooled to 60° C., were neutralized by adding 0.63 g of acetic acid and the reactor was emptied.

Analysis

OH number=99.1 mg KOH/g

pH (5% strength aqueous solution): 6.0

Cloud point (method E in accordance with DIN EN 1890): 76.2° C.

### Preparation Example 4

### Preparation of Hexahydrofarnesol-10 EO

A stirred reactor was charged with 175.6 g of hexahydrofarnesol and 1.25 g of a 44% strength aqueous KOH solution. The mixture was dewatered for 30 minutes at 120° C. and at 20 mbar. It was then rendered inert with nitrogen up to a pressure of 0.5 bar. At a temperature of 160° C. and a pressure of at most 3.0 bar, 339.2 g of ethylene oxide were metered in. When the metered addition was complete, the reactor contents, cooled to 60° C. were neutralized by adding 0.66 g of acetic acid and the reactor was emptied.

Analysis

OH number=80.9 mg KOH/g

pH (5% strength aqueous solution): 5.8

Cloud point (method E in accordance with DIN EN 1890): 84.7° C.

### Preparation Example 5

### Preparation of Hexahydrofarnesol-5.4 EO-2.0 PO

A stirred reactor was charged with 199 g of hexahydrofarnesol and 2.64 g of a 44% strength aqueous KOH solution. The mixture was dewatered for 30 minutes at 120° C. and 20 mbar. It was then rendered inert with nitrogen up to a pressure of 0.5 bar. At a temperature of 150° C. and a pressure of at most 2.3 bar, 206.9 g of ethylene oxide were metered in. After a post-reaction time of 15 minutes at 150° C., 101.1 g of propylene oxide were metered in at a temperature of 128° C. and a pressure of at most 2.9 bar. After a post-reaction time of 2.5 hours at 128° C., the reactor contents, cooled to 60° C., were neutralized by adding 1.26 g of acetic acid and the reactor was emptied.

Analysis

OH number=94.7 mg KOH/g

pH (5% strength aqueous solution): 6.4

Cloud point (method E in accordance with DIN EN 1890): 50.9° C.

### Preparation Example 6

### Preparation of Hexahydrofarnesol-1.2 PO-5.0 EO

A stirred reactor was charged with 182.4 g of hexahydrofarnesol and 1.7 g of a 44% strength aqueous KOH solution. The mixture was dewatered for 30 minutes at 120° C. and 20 mbar. It was then rendered inert with nitrogen up to a pressure of 1.0 bar. At a temperature of 130-140° C. and a pressure of at most 2.6 bar, 55.8 g of propylene oxide were metered in. After a post-reaction time of 15 minutes at 135° C., 176.2 g of ethylene oxide were metered in at a temperature of 163-173° C. and a pressure of at most 2.3 bar. After a post-reaction time of 40 minutes at 165° C., the reactor contents, cooled to 60° C., were neutralized by adding 0.8 g of acetic acid and the reactor was emptied.

Analysis

OH number=107.3 mg KOH/g

pH (5% strength aqueous solution): 6.1

Cloud point (method E in accordance with DIN EN 1890): 62.0° C.

### Preparation Example 7

### Preparation of Hexahydrofarnesol-4.5 PO-2.2 EO

A stirred reactor was charged with 159.6 g of hexahydrofarnesol and 2.34 g of a 44% strength aqueous KOH solution. The mixture was dewatered for 30 minutes at 120° C. and 20 mbar. It was then rendered inert with nitrogen up to a pressure of 1.0 bar. At a temperature of 125-135° C. and a pressure of at most 3.2 bar, 184.2 g of propylene oxide were metered in. After a post-reaction time of 15 minutes at 130° C., 68.8 g of ethylene oxide were metered in at a temperature of 145-155° C. and a pressure of at most 3.5 bar. After a post-reaction time of 55 minutes at 150° C., the reactor contents, cooled to 60° C., were neutralized by adding 1.1 g of acetic acid and the reactor was emptied.

Analysis

OH number=93.3 mg KOH/g

pH (5% strength aqueous solution): 7.0

Cloud point (method E in accordance with DIN EN 1890): 41.8° C.

### Preparation Example 8

### Preparation of Hexahydrofarnesol-15 PO

A stirred reactor was charged with 91.2 g of hexahydrofarnesol and 0.44 g of a 5% strength suspension of double metal cyanide catalyst (Zn/Co catalyst prepared as described in DE 100 08 629 A1, in the comparative example therein; using tridecanol N instead of PPG 400 for the suspension) in tridecanol N. The mixture was dewatered for 60 minutes at 120° C. and 20 mbar. It was then rendered inert with nitrogen up to a pressure of 0.5 bar. At a temperature of 150° C. and a pressure of at most 1.6 bar, 348.5 g of propylene oxide were metered in. When the metered addition was complete and after a post-reaction time of 30 minutes, the reactor contents, cooled to 80° C., were discharged.

Analysis

OH number=49.6 mg KOH/g

Cloud point (method E in accordance with DIN EN 1890): 8° C.

### C) Application Examples

### Application Example 1

### Shampoo

A shampoo formulation according to table 1 below was prepared.

TABLE 1

| | Amount [% by |
|---|---|
| Ingredient | weight] |
| Sodium laureth sulfate | 9.0 |
| Decyl glucoside | 2.0 |
| Cocoamidopropyl betaine | 1.0 |
| Hexahydrofarnesol-7 EO | 1.0 |
| Water | ad 100 |

### Application Example 2

### Hair Rinse

A hair rinse formulation according to table 2 below was prepared.

TABLE 2

| | Amount [% by |
|---|---|
| Composition | weight] |
| Cetearyl alcohol | 4.00 |
| Glyceryl stearate | 0.50 |
| Dicocoylethyl hydroxyethylammonium | 0.05 |
| methosulfate | |
| Propylene glycol | 2.50 |
| Glyceryl oleate | 0.70 |
| Hexahydrofarnesol-7 EO | 0.90 |
| Water | ad 100.0 |

### Application Example 3

### Liquid Detergent

A liquid detergent formulation with a composition according to table 3 was prepared.

TABLE 3

| | Amount |
|---|---|
| | [% by |
| Composition | weight] |
| Sodium fatty alkyl sulfate with a carbon chain distribution of | 10.00 |
| 1% by weight of C₁₀, 62% by weight of C₁₂, 23% by weight | |
| of C₁₄, 11% by weight of C₁₆, 3% by weight of C₁₈; the | |
| content of free fatty alcohol in fatty alcohol sulfate was | |
| less than 1% by weight | |
| Oleic acid, technical-grade | 6.50 |
| Coconut fatty acid (C₁₂-C₁₈) | 7.50 |
| Hexahydrofarnesol-7 EO | 6.00 |
| C₁₂-C₁₄-Alkyl glucoside (Degree of oligomerization x = 1.4) | 3.00 |
| Sodium hydroxide | 4.00 |
| Ethanol | 7.00 |
| 1,2-Propanediol | 6.00 |
| Citric acid (calculated as free acid) | 0.50 |
| Water and residual constituents | ad 100.00 |

### Application Example 4

### Washing Powder

A detergent formulation with a composition according to table 4 was prepared.

TABLE 4

| | Amount |
|---|---|
| | [% by |
| Composition | weight] |
| C₉-C₁₃-Alkyl benzosulfonate | 15.50 |
| Tallow fatty alcohol having on average 5 EO | 0.63 |
| Sodium perborate monohydrate | 20.00 |
| C₁₂-C₁₈-Sodium fatty soap | 0.80 |
| Polyethylene glycol 4000 | 5.00 |
| Sodium carbonate | 5.36 |
| Hexahydrofarnesol-7EO | 6.30 |
| Copolymeric sodium salt of acrylic acid and maleic acid | 2.28 |
| C_{12/18}-Fatty alkyl sulfate (92% by weight active substance, | 7.26 |
| 3.70% by weight of sodium sulfate, 2.8% by weight of other | |
| salts of raw materials and unsulfated fractions, 1.50% by weight | |
| of water) | |
| Zeolite A, based on anhydrous active substance | 22.52 |
| Fatty alcohol C_{12/14}-polyglycoside (78% by weight active | 5.00 |
| substance, 18% by weight of water glass modulus 2, 4.5% by | |
| weight of water | |
| Amorphous sodium silicate | 1.60 |
| Perfume oil | 0.45 |
| Water | 7.30 |

### Application Example 5

### Solublization of a Scent Oil in an Aqueous Surfactant Solution

Firstly, a surfactant solution of the following composition was prepared:

TABLE 5

| | Amount [% by |
|---|---|
| Component | weight] |
| Sodium laureth sulfate | 9.0 |
| Decyl glycoside | 2.0 |
| Cocoamidopropyl betaine | 1.0 |
| Water | ad 100 |

30 g of this surfactant solution were introduced as initial charge, and an amount of a mixture of 1 part by weight of tea tree oil and 4 parts by weight of the hexahydrofarnesol-7 EO was added to this until clouding of the solubilizate occurred.

Reference is made expressly to the disclosure of the documents cited herein.

1. A farnesol alkoxylate of the general formula I
R—O-(-A-O—)ₓ—H (I)
in which
R is an at least partially hydrogenated farnesyl radical,
A is identical or different low alkylene groups, where the polyoxyalkylene radical (-A-O)ₓ— can consist of one or more different oxyalkylene blocks, and
x is an integer from 1 to 100; or a mixture of such compounds.

2. The farnesol alkoxylate according to claim 1, in which R is a (C₁₅) radical of the formula II 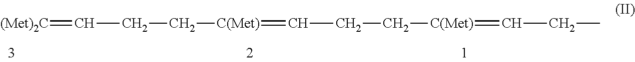
in which
one of the double bonds in positions 1, 2 or 3, or
two of the double bonds in positions 1, 2 or 3, or
all three double bonds in positions 1, 2 and 3,
are hydrogenated; or
in which R is a mixture of such partially or completely hydrogenated radicals, optionally additionally comprising non-hydrogenated farnesyl radicals.

3. The farnesol alkoxylate according to either of the preceding claims, in which
a) the group -(-A-O)ₓ— is a polyoxyalkylene radical
in which
x is a value from 3 to 50; and
A is in each case identical low alkylene groups;
or
A has different meanings and is at least two different low alkylene groups which are distributed randomly over the polyoxyalkylene radical; or
b) the polyoxyalkylene radical -(-A-O)ₓ— is composed of n oxyalkylene blocks -(-A₁-O)ₓ₁— to -(-Aₙ-O)ₓₙ—, where x is the sum x1+ . . . +xn and at least two oxyalkylene blocks differ with regard to the meaning of the alkylene radicals (A₁ . . . Aₙ) and/or with regard to the number of their monomer building blocks (x1 . . . xn) and where x1 to xn, independently of one another, are integers from 1 to 50.

4. The farnesol alkoxylate according to claim 3, in which the polyoxyalkylene radical is composed of different oxyalkylene blocks and is selected from the radicals:
-(PO)ₓ₁-(EO)ₓ₂-H
-(PO)ₓ₁-(EO)ₓ₂-(AlkO)ₓ₃-H
in which
PO is propylene oxide, EO is ethylene oxide and AlkO is C₃-C₁₀-alkylene oxide,
x1 is an integer from 1, 2 or 3;
x2 is an integer from 3 to 50; and
x3 is an integer from 1 to 10.

5. The farnesol alkoxylate according to claim 4, in which
x1 is an integer from 1 to 2;
x2 is an integer from 6 to 9, such as in particular 7 or 8; and
x3 is an integer from 1 to 5.

6. A process for the preparation of a compound of the general formula I
R—O-(-A-O—)ₓ—H (I)
where
a) an alcohol of the general formula III
R—OH (III)
in which R has the meanings given above, is alkoxylated in the presence of an alkoxylating catalyst, in the presence or absence of a solvent and also in the presence of at least one low alkylene oxide or low alkylene oxide mixture, giving a compound of the formula I,
in which the group -(-A-O)ₓ— is a polyoxyalkylene radical in which
x is a value from 4 to 50 and
A is in each case identical low alkylene groups; or
A has different meanings and is at least two different low alkylene groups which are distributed randomly over the polyoxyalkylene radical;
or
b) an alcohol of the general formula III
R—OH (III)
in which R has the meanings given above, is alkoxylated in the presence of an alkoxylating catalyst, in the presence or absence of a solvent and in the presence of a first low alkylene oxide, and the alkoxylation is repeated (n−1) times changing the low alkylene oxide, where n is an integer (such as e.g. from 1 to 10),
giving a compound of the formula I in which the polyoxyalkylene radical -(-A-O)ₓ— is composed of n oxyalkylene blocks -(-A₁-O)ₓ₁— . . . -(-Aₙ-O)ₓₙ— where x is the sum x1+ . . . +xn and at least two oxyalkylene blocks differ from one another with regard to the meaning of the alkylene radicals (A₁ . . . Aₙ) and/or with regard to the number of their monomer building blocks (x1 . . . xn) and where x1 to xn, independently of one another, are integers from 1 to 50.

7. The process according to claim 6b), where
i) the alcohol of the above formula III is propoxylated in the presence of a multimetal cyanide catalyst and in the presence or absence of a solvent and in the presence of propylene oxide to give a propoxylate of the formula IV
R—O—(PO)ₓ₁—H (IV)
and then
ii) the propoxylate formed in this way is further alkoxylated by adding one of the following blocks
-(EO)ₓ₂- or
-(EO)ₓ₂-(AlkO)ₓ₃-

8. The process according to claim 7, in which the multimetal cyanide catalyst is selected from catalysts which have at least one dimetal cyanide compound of the general formula (D)
M²ₐ[M¹(CN)ᵣXₜ]_{b}
in which
M¹ is a metal ion from the group consisting of Zn(II), Fe(II), Co(III), Ni(II), Mn(II), Co(II), Sn(II), Pb(II), Fe(III), Mo(IV), Mo(VI), Al(III), V(IV), V(V), Sr(II), W(IV), W(VI), Cu(II) and Cr(III),
M² is a metal ion from the group consisting of Sr(I), Mg(II), Zn(II), Fe(II), Fe(III), Co(III), Cr(III), Mn(II), Mn(III), Ir(III), Rh(III), Ru(II), V(IV), V(V), Co(II), Cr(II), Ti(IV),
X is a group different from cyanide which forms a coordinative bond to M¹, selected from the group consisting of carbonyl, cyanate, isocyanate, nitrite, thiocyanate and nitrosyl,
a, b, r, t are integers which are selected such that the electroneutrality condition is satisfied.

9. The process according to claim 8, where M¹ is Zn(II) and M² is selected from Co(III) and Fe(III).

10. A farnesol alkoxylate-containing reaction product obtainable by a process according to any one of claims 6 to 9.

11. The use of at least one compound according to any one of claims 1 to 5 or of a reaction product according to claim 10 in washing, rinsing, cleaning or finishing compositions; in cosmetic compositions; in fuels or fuel additive mixtures; in agrochemical compositions; or in compositions for papermaking.

12. A composition selected from washing, rinsing, cleaning and finishing compositions; cosmetic compositions; compositions for papermaking; agrochemical compositions; or fuels or fuel additive mixtures, comprising at least one compound according to any one of claims 1 to 5 or a reaction product according to claim 10.

13. A method, selected from washing, rinsing, cleaning or finishing methods; methods for papermaking; or methods for producing agrochemical compositions or fuels or fuel additive mixtures, where at least one compound according to any one of claims 1 to 5 or a reaction product according to claim 10 is used in this method.

14. The use of a compound according to any one of claims 1 to 5 or of a reaction product according to claim 10 as solubilizer in aqueous systems/liquids, such as in particular during emulsion polymerization.
